SEQUENCE LISTING (16 sequences)

SEQ ID NO: 1 - RNA, 80 nt, artificial
  40nt random RNA sequences flanked by two constant regions for amplification and in vitro transcription
gggagagacaagcuugggucnnnnnnnnnnnnnnnnnnnnnnnnnnnnnnnnnnnnnnnncucuugcucuuccuaggagu

SEQ ID NO: 2 - DNA, 20 bp, artificial
  3′ blocking ssDNA for selection
actcctaggaagagcaagag

SEQ ID NO: 3 - DNA, 20 bp, artificial
  5′ blocking ssDNA for selection
gacccaagcttgtctctccc

SEQ ID NO: 4 - DNA, 39 bp, artificial
  RT-PCR forward primer for selection
tctaatacgactcactatagggagagacaagcttgggtc

SEQ ID NO: 5 - DNA, 20 bp, artificial
  RT-PCR reverse primer for selection
actcctaggaagagcaagag

SEQ ID NO: 6 - DNA, 31 bp, artificial
  amplification primer with BamHI restriction site
gcttggatcctctaatacgactcactatagg

SEQ ID NO: 7 - DNA, 30 bp, artificial
  amplification primer with EcoRI restriction site
ggtcgaattcactcctaggaagagcaagag

SEQ ID NO: 8 - RNA, 80 nt, artificial
  Aptamer 1
gggagagacaagcuuggguccaugccacucuacaccucacucacgggaaucacacaccgccucuugcucuuccuaggagu

SEQ ID NO: 9 - RNA, 80 nt, artificial
  Aptamer 2
gggagagacaagcuugggucgcauaucaguauuccacgugacuguuaccuaccucuaccgcucuugcucuuccuaggagu

SEQ ID NO: 10 - RNA, 80 nt, Artificial
  Aptamer 3
gggagagacaagcuuggguccaaccgcccuuaccucgcaaacauauucgaagucccacugcucuugcucuuccuaggagu

SEQ ID NO: 11 - RNA, 80 nt, Artificial
  Aptamer 4
gggagagacaagcuugggucccuacagcugcaauugcccuaaaccuucacgcuggccguccucuugcucuuccuaggagu

SEQ ID NO: 12 - RNA, 80 nt, Artificial
  Aptamer 5
gggagagacaagcuugggucagacauagcuaccgcauuuucauccuccauuugccgcagccucuugcucuuccuaggagu

SEQ ID NO: 13 - RNA, 79 nt, Artificial
  Aptamer 6
ggagagacaagcuugggucggaacauguucgucauuuguuacuacuuuacgacugccugcucuugcucuuccuaggagu

SEQ ID NO: 14 - RNA, 80 nt, Artificial
  Aptamer 7
gggagagacaagcuuggguccaacucuauuauacacgguccgauacugaccacauuugcccucuugcucuuccuaggagu

SEQ ID NO: 15 - RNA, 80 nt, Artificial
  Aptamer 8
gggagagacaagcuuggguccuaccacaacucacugcaccacgucuaccuugccauugaccucuugcucuuccuaggagu

SEQ ID NO: 16 - protein, 12 aa, Artificial
  Peptide S186
AKPYSQRRKTSG

(19) United States
(12) Patent Application Publication
(10) Pub. No.: US 2009/0092980 A1
(43) Pub. Date: Apr. 9, 2009

(54) miRNA PROCESSING INHIBITOR EFFICACY ASSAYS AND SUBSTANCES

(76) Inventors: Christoph Arenz, Berlin (DE); Brian Patrick Davies, Berlin (DE); Saskia Neubacher, Berlin (DE); Claudine Mercedes Klemm, Berlin (DE)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/176,122

(22) Filed: Jul. 18, 2008

Related U.S. Application Data
(60) Provisional application No. 60/961,366, filed on Jul. 20, 2007.
(60) Provisional application No. 61/034,768, filed on Mar. 7, 2008.

(51) Int. Cl. C12Q 1/68; C07H 21/02; C07H 15/26

(52) U.S. Cl. 435/6; 536/23.1; 536/17.4

(57) ABSTRACT

The invention relates to assays for assessing miRNA maturation effector (preferably: inhibitor) efficacy, and to substances useful for influencing, particularly for inhibiting, maturation of miRNA. According to the invention there is provided assay of miRNA processing inhibitor efficacy, comprising the steps of: a) providing a target miRNA precursor, b) providing a potential inhibitor of one or more processing steps of the target miRNA precursor, c) bringing together of the target miRNA precursor and the potential inhibitor under miRNA maturation conditions, and d) determining inhibition efficiency. The assay of the present invention allows for a very fast and easy assessment of the efficacy of a potential inhibitor in inhibiting processing of a miRNA precursor into miRNA.

This application claims priority to U.S. Application Nos. 60/961,366 and 61/034,768 and incorporates them by reference.

### BACKGROUND OF THE INVENTION

1. Field of the Invention

The invention relates to assays for assessing miRNA maturation effector (preferably: inhibitor) efficacy, and to substances useful for influencing, particularly for inhibiting, maturation of miRNA.

2. Description of Related Art

MicroRNAs (miRNAs) are small, regulatory RNAs. They are endogenous in a wide variety of eukaryotic cells, especially mammalian and, in particular, murine and human cells. Generally, miRNAs exert their regulatory properties by protein mediated binding to messenger RNA (mRNA) such as to inhibit translation of the respective mRNA and corresponding protein expression. miRNAs are first transcribed as long primary transcripts (primary precursors) in the nucleus. Drosha nuclease cleaves from these primary transcripts approximately 70-75 nt precursors (pre-miRNAs). Theses miRNA precursors are regulatorily still inactive. They are exported from the nucleus and are further cleaved in the cytoplasm by the RNase III enzyme Dicer. This process of miRNA precursor processing, particularly the processing of pre-miRNAs by Dicer, is termed miRNA maturation. The resulting miRNAs are approximately 21 nt double-stranded RNAs and are bound by a number of proteins to produce a ribonucleic acid protein complex (RNP). In such complexes, miRNA is unwound to build a miRNA induced silencing complex (miRISC). This miRISC can then bind, directed by the guide strand of miRNA, to a corresponding target mRNA, inhibiting the translation of said target mRNA. For a general overview of miRNA processing, a skilled person will consider the following documents, which are incorporated herein by reference in their entirety: E. G. Moss et al., Curr. Biol. 2002, R688-R690 “MicroRNAs, something new under the sun”; A. Grishok et al., Cell 2001, 23-34 “Genes and mechanisms related to RNA interference regulate expression of the small temporal RNAs that control C. elegans developmental timing”; G. Hutvagner et al., Science 2001, 834-838 “A cellular function for the RNA-interference enzyme Dicer in the maturation of the let-7 small temporal RNA”.

Generally, miRNA processing and the role of miRNAs in cell and organ development and metabolism are not well understood. However, a number of studies suggest a role for miRNAs in a wide range of functions in mammals, particularly the development of brain, heart and skeleton muscle and insulin secretion. Further evidence suggests a role for miRNAs in the development of diseases like cancer and for susceptibility to viral infections. Those miRNAs that have been shown or that are suspected of being able to promote cancer, like miR-21, miR-29-b2, miR-211, miR-18, miR-224, miR-17-92, miR-155, miR-221, miR-222, miR-146, miR-181, miR-372, miR-373, miR-141, miR-200b, miR-10a, miR-20a, miR-24-1, miR-31, miR-96, miR-183, miR-197, miR-346, miR-224, miR-205, miR-210, miR-103-2, miR-223, miR-203, miR1-195 and others have been termed “oncomirs” in analogy to the well-known “oncogenes”. A skilled person will consider the following documents, which are incorporated herein by reference in their entirety: D. Zhang et al. Developmental Biology 2007, 1-12 “microRNAs as oncogenes and tumor suppressors”; E.A.C. Wiemer European Journal of Cancer 2007, 43, 1529-1544 “The role of microRNAs in cancer: no small matter” Other miRNAs have been identified that seem to protect cells against malign transformation. It is presently believed that up to 1,000 different miRNAs can be present in human cell types (E. Berezikov et al., Cell 2005, 21). The elucidation of miRNA processing and their function in cells and organs is thus of considerable interest.

One approach so far has been to block the effects of miRNAs in cells by exposing cells to antisense molecules. The antisense molecules, particularly DNA or RNA strands with or without modifications, are intended to bind to the miRNA and particularly its guide strand to inhibit formation of miRISC or binding to the miRISCs target mRNA. Generally, these approaches are hampered by the cell membrane's barrier function, which does not readily allow antisense molecules to enter the cytoplasm. To overcome this problem, RNAs termed “antagomirs” have been tried with some success. Antagomirs are RNA-like oligonucleotides that comprise various modifications for RNase protection and for enhanced tissue and cell uptake. They differ from normal RNA inter alia by 2′-O-methylation of the ribose, a phosphorothioate backbone and a cholesterol-moiety at the 3′-end (cf. J. Krützfeldt et al., Nucl. Acids Res. 2007, 2885-2892, “Specificity, duplex degradation and subcellular localization of antagomirs”.

However, present approaches still require that each antagomir be individually developed. Because of their medications, it is so far not feasible to produce a high number of antagomirs and test their influence on miRNA maturation or miRNA functioning in cells and tissues in a high throughput format.

### BRIEF SUMMARY OF THE INVENTION

The inventors have now found that for understanding and manipulation of miRNA maturation, pre-miRNA cleavage by Dicer is a particularly valuable entry point. It was thus the problem of the invention to provide an assay for fast and easy assessment of miRNA processing inhibitor efficacy, reagents useful in said assay and corresponding miRNA processing inhibitors.

According to the invention there is provided an assay of miRNA processing inhibitor efficacy, comprising the steps of:
a) providing a target miRNA precursor,
b) providing a potential inhibitor of one or more processing steps of the target miRNA precursor,
c) bringing together of the target miRNA precursor and the potential inhibitor under miRNA maturation conditions, and
d) determining inhibition efficiency.

The assay of the present invention allows for a very fast and easy assessment of the efficacy of a potential inhibitor in inhibiting processing of a miRNA precursor into miRNA.

A target miRNA precursor can be a primary precursor or, preferably, a pre-miRNA. The target miRNA precursors can comprise modifications to allow or facilitate detection of processing of said precursor to a miRNA. Such modifications can include radioactive markers, e.g. in miRNA precursor sections that are not retained in the final miRNA, such that loss of radioactivity in a sample of nucleotides with the expected size of the respective miRNA indicates miRNA processing. Preferably, if a modification is applied for detection, the modification comprises a color or fluorescence-generating moiety. Particularly preferred is thus a miRNA precursor, particularly a pre-miRNA, comprising a fluorescence emitter at one end (e.g. the 5′-end), and a fluorescence quencher on the other end (e.g. the 3′-end). Due to intramolecular nucleotide pairing the fluorescence emitter of one end and the fluorescence quencher of the other end of the target miRNA precursor are brought into close contact with one another, so that fluorescence is quenched for the unprocessed target miRNA precursor. Upon cleavage of the target miRNA precursor, particularly a target pre-miRNA, the fluorescence emitter and the fluorescence quencher become dissociated from one another leading to an increase in fluorescence indicative of miRNA processing.

For the analysis of Dicer mediated miRNA processing, it is particularly preferred that the target miRNA precursor has a length of 60-80 nt, even more preferred 60-70 nt. Furthermore, it is preferred that the target contain an overhang of 2 nucleotides at the 3′-end. The target may also contain an overhang of 1 nucleotide at the 3′-end or even a blunt end structure whereby there are no unpaired 3′-terminal nucleotides in the target miRNA precursor. The structure containing an overhang of 2 nucleotides at the 3′-end is cleaved most effectively (FIG. 3). For details, the skilled person will consider the publications B. P. Davies et al., Angew. Chem. 2006, 5676-5679 “Ein homogener Assay der microRNA-Reifung”, or alternatively B. P. Davies et al., Angew. Chem. Int. Ed. Engl. 45, 5550-5552 “A Homogenous Assay for Micro RNA Maturation” and B. P. Davies and C. Arenz Bioorg. Med. Chem. (2007), doi: 10.1016/j.bmc.2007.04.055 “A Fluorescence Probe for Assaying micro RNA Maturation” which are incorporated herein in their entirety.

Further modifications of nucleic acid molecules are known to enhance RNA nuclease stability and efficacy. Such modifications can be particularly advantageous for the target miRNA precursor, but also for nucleic acid and particularly RNA aptamers, as detailed below. For example, to increase stability and/or enhance activity, oligonucleotides can be modified with nuclease resistant groups, particularly LNA, 2′-amino, 2′-C-allyl, 2′-fluoro, 2′-O-methyl, 2′-O-allyl and 2′-H. Sugar modifications of nucleic acid molecules have also been extensively described in the art. Particularly preferred modifications are those of phosphorothioate, of phosphorodithioate and/or 5-methylphosphonate modifications. For details, a skilled person can refer to WO 2005/021800 A2, particularly pages 47-53, which is incorporated herein by reference in its entirety. Particularly preferred modifications are furthermore those of LNA and PNA modifications that lead to improved binding to the target miRNA precursors.

According to the invention, a potential inhibitor is used in step b) of the assay according to the invention preferably is an aptamer or a peptide or a peptide derivative or a peptide mimic or a polyamine or a aminoglycoside or a aminoglycoside derivative or a bulge binder or a multimerized 2-deoxystreptamine or another small molecule. Further details are indicated below.

In step c) of the assay of the present invention, the target miRNA precursor, especially a (fluorescently) labelled pre-miRNA as described above, is brought into contact with the potential inhibitor under conditions that, in the absence of an inhibitor, allow processing of the target miRNA precursor into miRNA. Particularly preferred, the target miRNA precursor is a target pre-miRNA, and the processing conditions comprise providing an enzyme with Dicer functionality for pre-miRNA cleavage.

The inhibitors efficacy can be determined e.g. by detecting the rate of target miRNA maturation. Preferably, when the target miRNA precursor is labelled with a fluorogenic emitter label and a quenching moiety, the development for fluorescence indicates target miRNA precursor processing. Efficient inhibitors of target miRNA processing will then lead to the absence of or a slow increase fluorescence overtime, while less efficient inhibitors will allow for a faster development of a fluorescence signal. Optionally, the efficacy of a potential inhibitor can be compared against the efficacy of another potential inhibitor or against the efficacy of a standard inhibitor. Optionally, the target miRNA precursor is labelled with two different fluorogenic emitter labels comprising a FRET-donor and a FRET-acceptor, the change of the fluorescence intensities at two different wavelengths indicates target miRNA precursor processing.

It is particularly preferred to apply steps a) to d) to each of a plurality of potential inhibitors. The assay of the present invention is particularly suitable for implementation in a high throughput format and thus allows screening large libraries of potential inhibitors. It is particularly preferred to perform steps a) to d) in a well of a microtitre plate for each potential inhibitor tested, thus allowing up to e.g. 96 or 384 or 1536 potential inhibitors to be assayed on one microtiter plate.

According to the invention, the target miRNA precursor is preferably selected from the group consisting of labelled or unlabelled pre-miRNA, or labelled or unlabelled pri-miRNA or labelled or unlabelled mirtrons. Mirtrons are miRNA precursors that bypass Drosha processing. A skilled person will consider the following documents, which are incorporated herein by reference in their entirety: J G Ruby et al. Nature 2007, 448, 83-6 “Intronic miRNA precursors that bypass Drosha processing”; K. Okamura et al. Cell 2007, 130, 89-100 “The Mirtron Pathway Generates microRNA-Class Regulatory RNAs in Drosophila”.

Particularly for pre-miRNAs or Mirtrons as target miRNA precursors, it is preferred that the processing conditions step c) comprise provision of an enzyme with Dicer functionality. The cleavage of pre-miRNA or a Mirtron to produce miRNAs is a decisive step in miRNA processing and particularly valuable for influencing cellular miRNA processing. It is particularly preferred to develop inhibitors of pre-miRNA processing by Dicer, since such inhibitors only have to be brought into a cytoplasm and need not further be transported into the nucleus, as e.g. would be necessary for inhibitors of Drosha processing.

Accordingly, it is particularly preferred when the processing conditions of step c) comprise provision of a cell expressing a miRNA processing enzyme, particularly preferred Dicer or an enzyme with Dicer functionality, or a respective cell extract or lysate. Such assays are easy to set up and allow assessment of potential inhibitor efficacies under conditions closely resembling those of a living tissue or a patient to be treated with miRNA processing inhibitors.

As indicated above, it is also preferred to compare the inhibition efficacy of a potential inhibitor (as determined in step d) of the assay of the present invention) with the corresponding efficacy of a standard inhibitor. Particularly preferred, such standard inhibitor is an aptamer, preferably an RNA aptamer, comprising, consisting essentially or consisting of any of sequences SEQ ID NO. 8 to 15 These aptamers have surprisingly proved themselves to be particularly good inhibitors of Dicer processing of D. melanogaster pre-let-7 pre-miRNA.

If the potential inhibitors are nucleic acids, e.g. optionally modified RNAs or DNAs, preferred assays of the present invention further comprise the steps of
e) selecting a potential inhibitor,
f) providing a further plurality of potential inhibitors with mutated nucleotide sequence or with modified backbone such as in the case of LNA or PNA or phosphorothioates.
g) repeating steps a) to d) for each of the further plurality of potential inhibitors.

The potential inhibitor is preferably selected as described above in step d), e.g. against a preselected standard inhibitor. The selected inhibitor is then mutated or in case of a multimerized RNA binder the linker structure is modified or in case of another small molecule, one or more functional groups are introduced or substituted to create a plurality of further potential inhibitors with a structure slightly deviating from the structure of the selected potential inhibitor. For each of the potential inhibitors of the further plurality, inhibition efficacy is then determined in an assay as described above. This way, inhibition efficacy and inhibition selectivity can be further improved by each round of steps a) to d).

According to the invention, the potential inhibitor is preferably a peptide or a peptide mimic or a peptoid or a polyamine or an aminoglycoside or a aminoglycoside derivative or an aptamer or a bulge binder. The advantages of such molecules are better cell permeability, higher potency, better ADME parameters and lower manufacturing costs in comparison to antisense molecules targeting the mature miRNA.

According to the invention, a miRNA maturation inhibitor is preferably an aptamer comprising, consisting essentially or consisting of a nucleotide of SEQ ID NO. 8 to 15 or a nucleotide sequence having at least 70% sequence homology to any of said nucleotide sequences. “Sequence homology” regarding nucleic acids according to the invention is determined according to the EMBOSS::needle (global) program with the following parameters: Gap open: 10.0; Gap extend: 0.5; Molecule: RNA; Matrix: DNAfull). The program implements the alignment algorithm of Needleman and Wunsch, cf. J. Mol. Biol. 1970, 443-453.

Also according to the invention, there is provided a miRNA processing inhibitor, comprising, consisting essentially or consisting of a peptide of SEQ ID No 16 or a peptide sequence having at least 70% sequence homology to said peptide. “Sequence homology” regarding proteins and peptides according to the invention is determined according to the EMBOSS::needle (global) program with the following parameters: Gap open: 10.0; Gap extend: 0.5; Molecule: PROTEIN; Matrix: Blosum62). The program implements the alignment algorithm of Needleman and Wunsch, cf. J. Mol. Biol. 1970, 443-453.

According to the invention, there are thus also provided:
a substance according to claim 12,
miRNA maturation inhibitors according to claim 13 and 14, and
assays for determining miRNA maturation effector efficacy according to claim 15.

The miRNA maturation inhibitors of formula (1)-(9) are hereinafter also termed “conjugate 1”-“conjugate 9”, respectively. The assay of the present invention allows for a very fast and easy assessment of the efficacy of a potential maturation effector, particularly a miRNA maturation inhibitor, in effecting (preferably: inhibiting) processing of a miRNA precursor into miRNA.

### BRIEF DESCRIPTION OF THE DRAWINGS

The invention is further described with respect to the figures and examples, without limiting the scope of the claims.

FIG. 1 Fluorescence increase upon incubation of 0.5 U recombinant Dicer with 20 nM D. melanogaster pre-let-7 pre-miRNA probe alone ( solid line) or in the presence of 100 μM kanamycin A (▪ solid line). Controls contain heat denatured recombinant Dicer with (▪ dotted line) or without ( dotted line) 100 μM kanamycin A. Further conditions: 20 mM Tris-HCl pH 7.4, 12.5 mM NaCl, 2.5 mM MgCl₂, 1 mM DTT in 40 μL in 384-well plate.

FIG. 2 Fluorescence increase upon incubation of 0.5 U recombinant Dicer with 20 nM D. melanogaster pre-let-7 pre-miRNA probe alone () or in the presence of 100 μM peptide S186 (▪). Assay conditions: 20 mM Tris-HCl pH 7.4, 150 mM NaCl, 2.5 mM MgCl₂, 1 mM DTT in 40 μL in 384-well plate.

FIG. 3 Comparison of Dicer cleavage of human miR-142 precursor containing either a 2 nucleotide overhang (solid line ▪) or 1 nucleotide overhang (dashed line ) with 5′-FAM as fluorophore and 3′-Dabcyl as quencher. Conditions: 40 μL containing 20 nM beacon, 20 mM Tris-HCl pH 6.8, 12.5 mM NaCl, 2.5 mM MgCl₂, 1 mM DTT and 1 U Dicer (Ambion) in 384-well microtiter plate.

FIG. 4 Dicer cleavage of human miR-19b-2 precursor containing 1 nucleotide overhang with 5′-Cy3 as fluorophore and 3′-Dabcyl as quencher. Conditions: 40 μL containing 100 nM beacon, 20 mM Tris-HCl pH 6.8, 12.5 mM NaCl, 2.5 mM MgCl₂, 1 mM DTT, 0.05% Tween 20 and 0.5 U Dicer (Ambion) in 384-well microtiter plate.

FIG. 5 Dicer cleavage of D. melanogaster Bantam precursor containing 2 nucleotide overhang with 5′-FAM as fluorophore and 3′-Dabcyl as quencher. Conditions: 40 μL containing 20 nM beacon, 20 mM Tris-HCl pH 6.8, 12.5 mM NaCl, 2.5 mM MgCl₂, 1 mM DTT 0.1 U Dicer (Invitrogen) in 384-well microtiter plate.

FIG. 6 Fluorescence increase upon incubation of 0.1 U recombinant Dicer with 20 nM D. melanogaster pre-let-7 pre-miRNA probe alone or in the presence of 100 nM aptamer or in the presence of 100 nM tRNA, 20 mM Tris-HCl pH 6.8, 12.5 mM NaCl, 2.5 mM MgCl₂, 1 mM DTT in 40 μL in 384-well plate, 0.1 U recombinant Dicer (Invitrogen).

FIG. 7 Inhibition of Dicer cleavage of the pre-let7 pre-miRNA probe at aptamer or tRNA concentrations of 100 nM

FIG. 8 Inhibition of Dicer cleavage of the pre-let7 pre-miRNA probe by other aptamers at 100 nM

FIG. 9 shows a way to synthesize 2-deoxystreptamine conjugates,

FIG. 10 shows inhibition of let-7 maturation by substances (1)-(9) at varying concentrations of these substances,

FIG. 11 shows a two-dimensional structure of the let-7 miRNA precursor as computed with m-fold,

FIG. 12 shows results of fluorescence measurements for miRNA maturation inhibitors (1)-(9) as detailed below, and

FIG. 13 shows Biacore-measurements for miRNA maturation inhibitors (1)-(9) as detailed below.

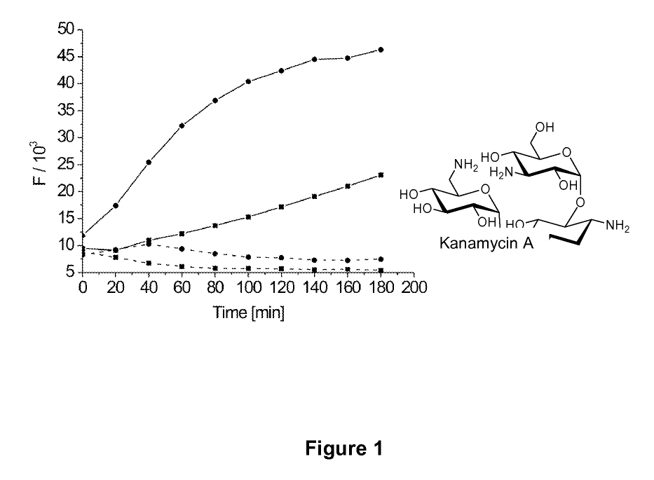

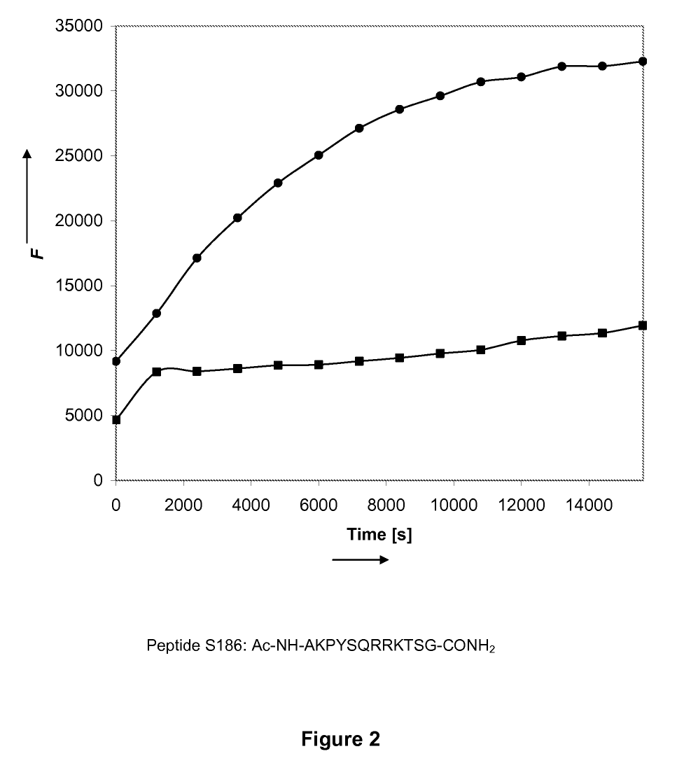

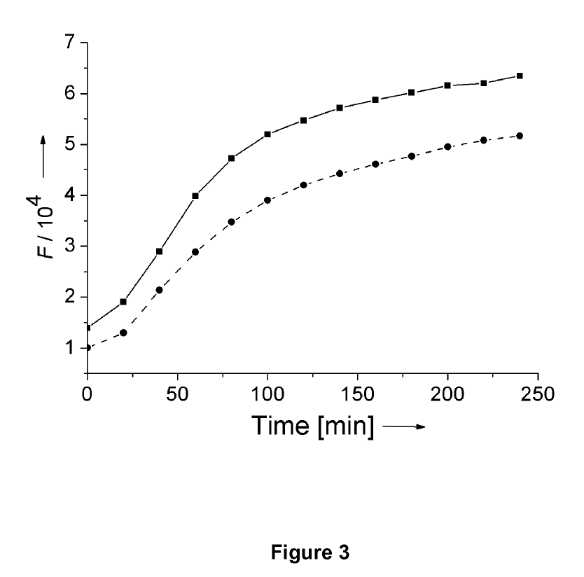

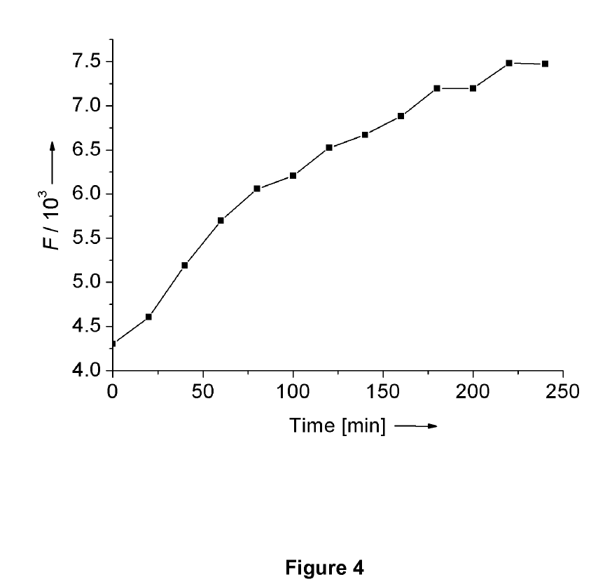

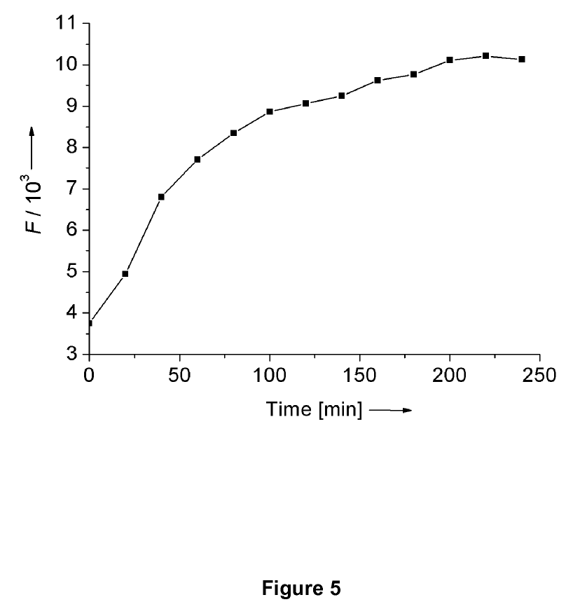

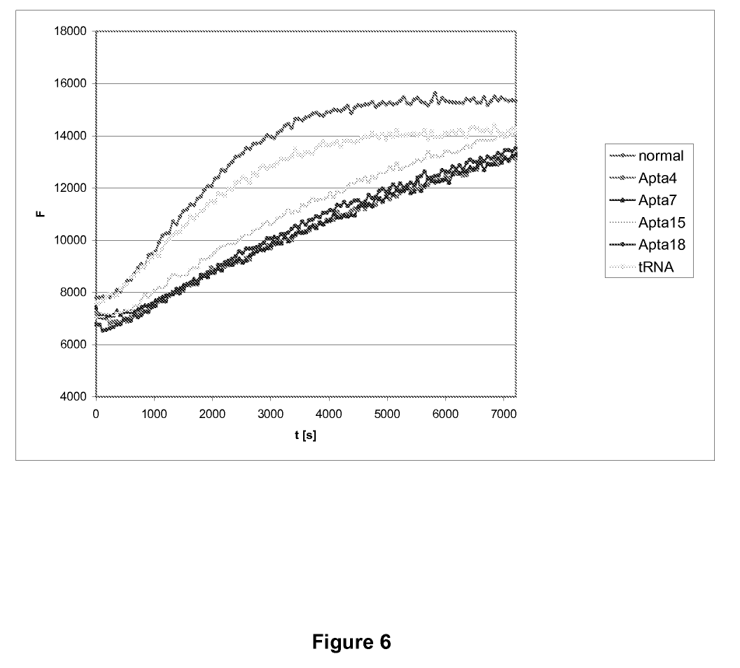

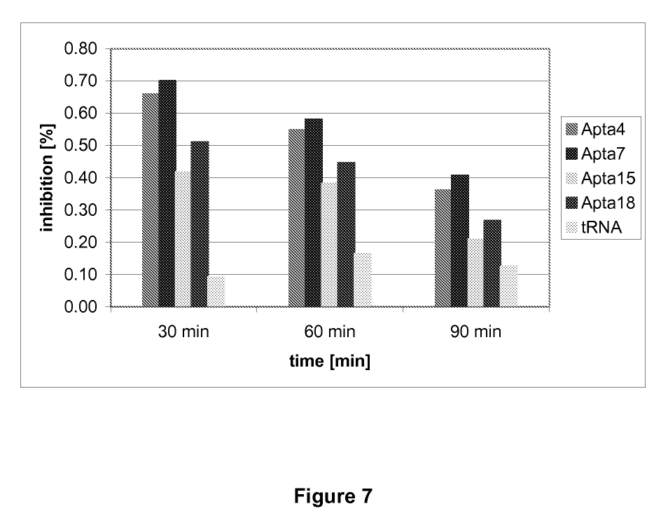

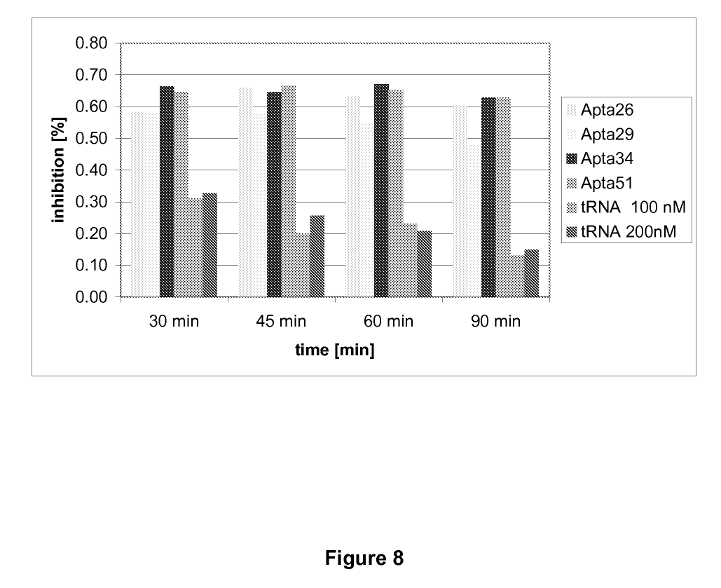

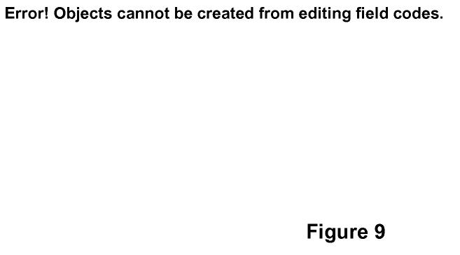

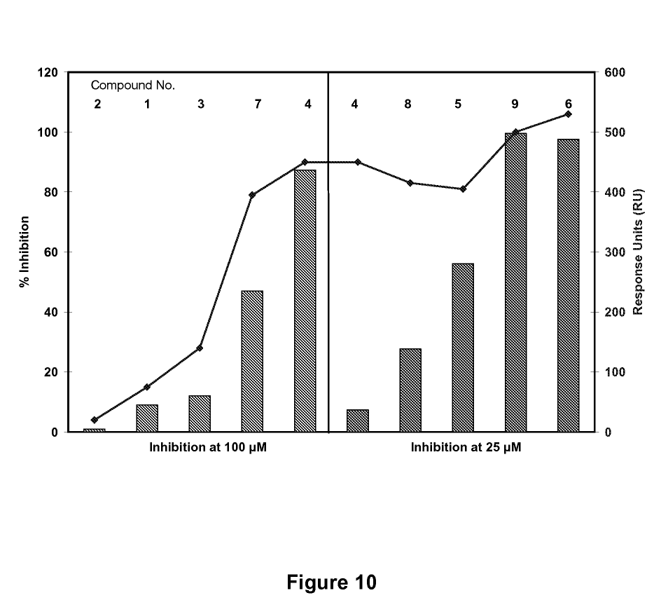

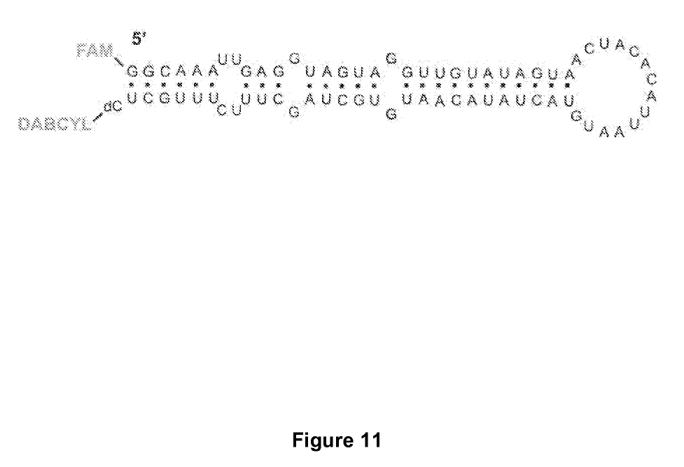

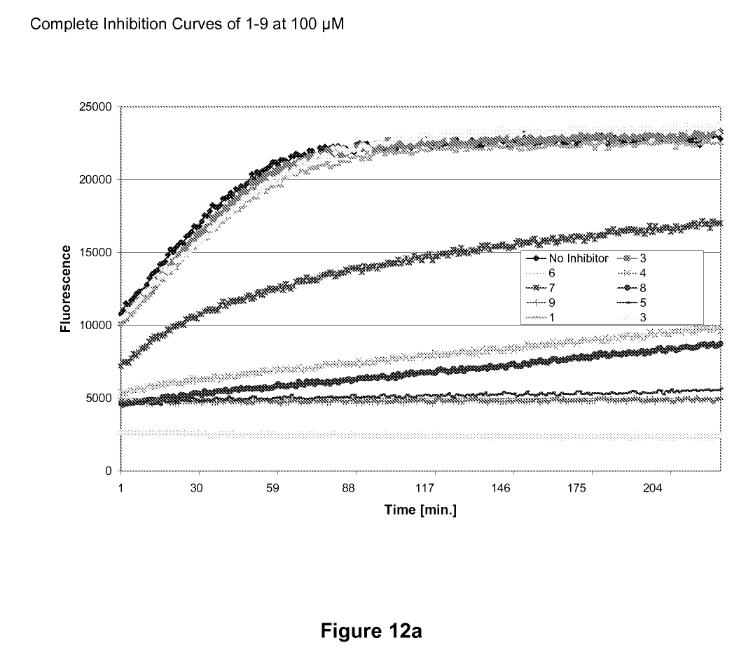

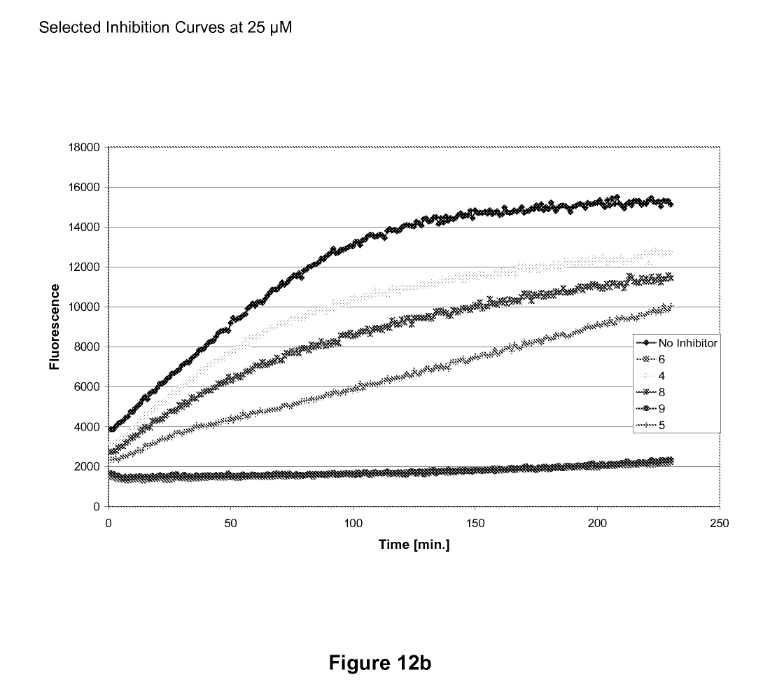

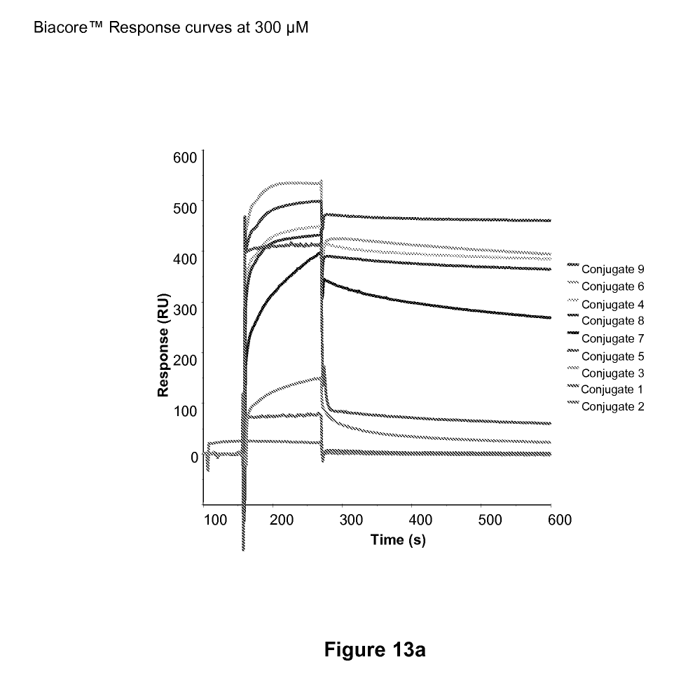

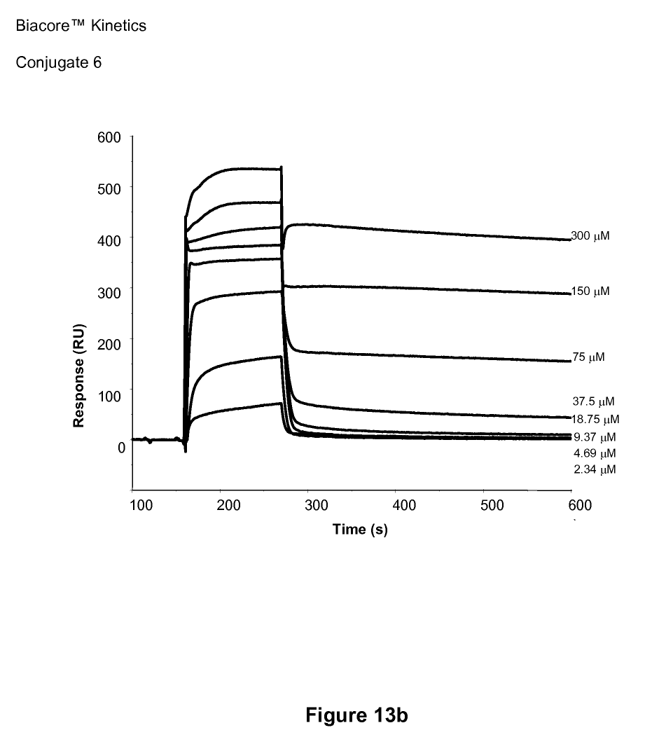

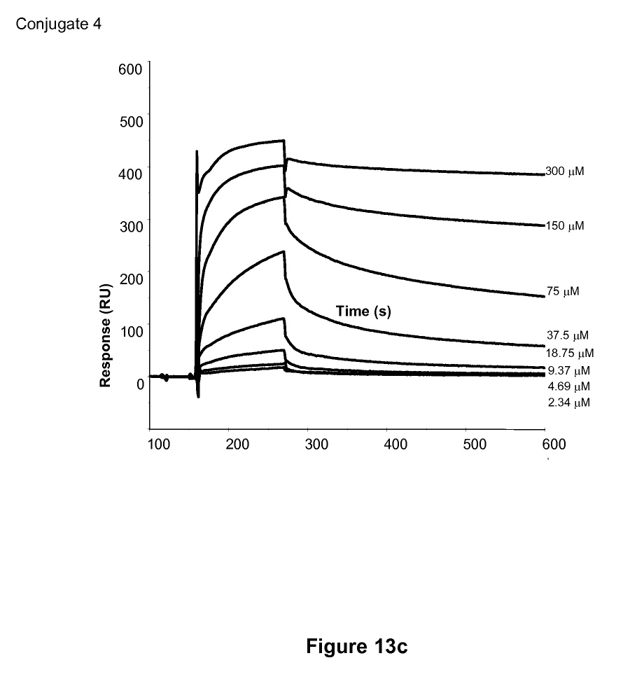

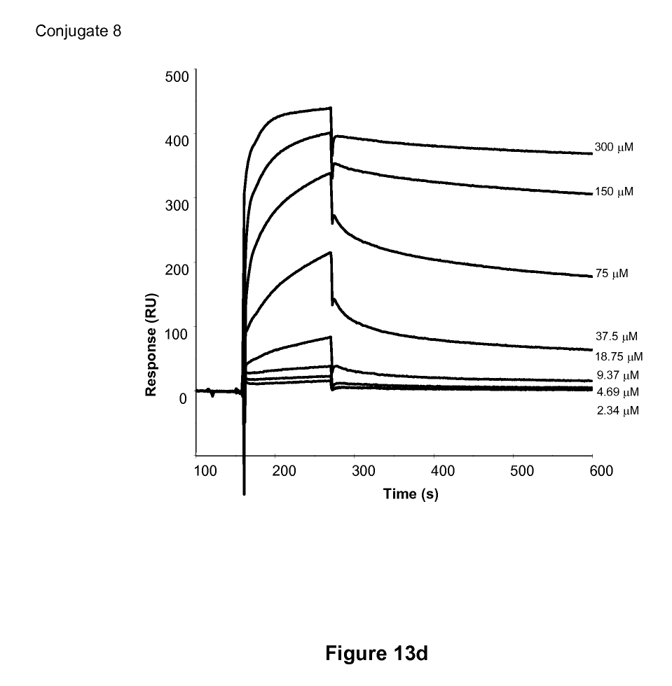

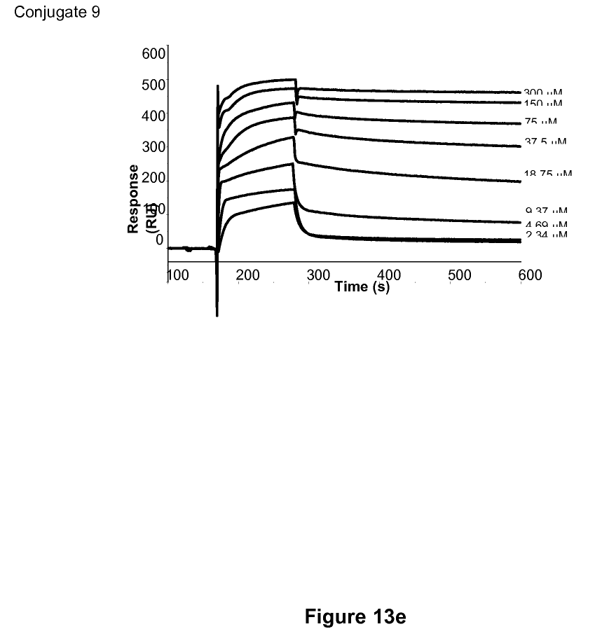

### DETAILED DESCRIPTION OF THE INVENTION

### Assay Conditions for miRNA Maturation Assay

The miRNA processing assay was performed in 96- or 384-well plates. For 384-well plates a final volume of 40 μL was used. An optimized 40 μL reaction contained 10-20 nM pre-miRNA probe, 20 mM Tris-HCl, pH 6.8, 12 mM NaCl, 2.5 mM MgCl₂, and 1 mM DTT. Kanamycin A, for example, was pre-incubated at room temperature for 30 min. Dicer was then added, either 0.25 U commercial recombinant Dicer or 10% HEK293 cell lysate, and the fluorescence increase measured every minute for 4 hours.

### HEK293 Cell Lysate Preparation

Human embryonic kidney cells (HEK293) were cultured in Dulbecco's modified eagle medium (Gibco, Invitrogen). The cells were trypsinated, collected, and centrifuged at 2,000 g and 4° C. for 5 min. The supernatant was discarded and the pellet washed once with phosphate buffered saline and again centrifuged at 2,000 g and 4° C. for 5 min. The supernatant was discarded and the pellet taken up in buffer containing 20 mM Tris-HCl, pH 7.4, 75 mM NaCl, 5 mM MgCl₂, 2 mM DTT, 10% glycerol and Roche protease inhibitor cocktail. The cells were lysed by ultrasound and centrifuged once at 4,300 g and 4° C. for 10 min. The supernatant was then transferred to a fresh reaction vial and centrifuged again at 12,100 g for 5-10 min at room temperature. The supernatant was transferred once again to a fresh reaction vial and stored at −20° C. until further use. Total protein concentration was determined by the Bradford assay and contained between 100 and 250 μg/mL.

For the analysis of Dicer mediated miRNA processing, it is particularly preferred that the target miRNA precursor has a length of 60-80 nt, even more preferred 60-70 nt. Furthermore, it is preferred that the target contain an overhang of 2 nucleotides at the 3′-end. The target may also contain an overhang of 1 nucleotide at the 3′-end or even a blunt end structure whereby there are no unpaired 3′-terminal nucleotides in the target miRNA precursor. The structure containing an overhang of 2 nucleotides at the 3′-end is cleaved most effectively.

### In Vitro Selection Protocol

For both D. Melanogaster pre-let-7 pre-miRNA and Bantam pre-miRNA the starting diversity was approx. 10¹⁵ RNA molecules, all consisting of 80 nt with a random sequence of 40 nt flanked by two constant regions for amplification and in vitro transcription (5′-GGGAGAGACAAGCUUGGGUC-N40-CUCUUGCUCUUCCUAGGAGU-3′). Before each round of selection 5′-biotinylated pre-miRNAs and RNA pools including an equimolar amount of 5′-blocking ssDNA (5′-GACCCAAGCTTGTCTCTCCC-3′) and 3′-blocking ssDNA (5′-ACTCCTAGGAAGAGCAAGAG-3′) were renatured by heating to 95° C. for 5 min and cooling to room temperature within 30 min in buffer A (20 mM HEPES, pH 7.3, 20 mM NaOAc, 140 mM KOAc, 3 mM MgCl₂). The first rounds were further preceded by a preselection round in which the renatured RNA pool was incubated with 180-50 ug (36-10 uL of a 5 mg/mL solution) of streptavidin-coated magnetic beads, depending on the selection round. The supernatant was then incubated for 25 min at room temperature with the according 5′-biotinylated pre-miRNA. 10-20 uL streptavidin-coated magnetic beads were added to the mixture and incubated for 5 min. The supernatant was removed and the beads were washed once to twice with selection buffer. Bound RNA molecules were eluted three times in 50 uL of water at 80° C. Beginning with selection round S5 the preselection was replaced by a counter selection. In the case of the pre-let-7 selection, the renatured RNA pool was first incubated for 25 min with 5′-biotinylated Bantam, then streptavidin-coated magnetic beads were added. The supernatant was then incubated with 5′-biotinylated pre-let7. For the Bantam selection the counter selection was performed with 5′-biotinylated pre-let-7. The pre-miRNA in the counter selection step can be varied or even several different pre-miRNAs can be used simultaneously or consecutively. Except for the first few selection rounds, incubation volumes were usually 100 uL. At selection round S3 in the pre-let-7 pre-miRNA selection and S7 in the Bantam selection rounds were proceeded with and without an incubation with nuclease Dicer. Following the selection, where RNA molecules are bound to the target 5′-biotinylated pre-miRNA 0.5-1 U of Dicer were added to the mixture and incubated for 1 to 1.5 h at 37° C. Streptavidin-coated magnetic beads were added, washed and eluted as described above. For further details concerning the selection rounds for pre-let-7 and Bantam see tables below. A total of 10 selection rounds were performed for both pre-miRNAs with and without Dicer cleavage. In case of a selection with Dicer buffer B (20 mM Tris-HCl, pH 6.8, 12.5 mM NaCl, 2.5 mM MgCl₂, 1 mM DTT) was used to ensure full Dicer activity.

Selection for pre-let-7 aptamers.

| selection | RNA-Pool | 5′-bio pre- | incubation | wash | |
|---|---|---|---|---|---|
| round | [uM] | let-7 [uM] | volume | [100 uL] | counter selection |
| S1 | 10 | 0.3 | 600 | 1 | streptavidin |
| S2 | 6.6 | 0.3 | 300 | 1 | streptavidin |
| S3 | 3 | 0.3 | 300 | 2 | streptavidin |
| S3 + Dicer | | | 100 | | 5 U Dicer 1 h |
| S4 | 1 | 0.15 | 150 | 2 | streptavidin |
| S4 + Dicer | | | | | 4 U Dicer 2 h |
| S5 | 1 | 0.15 | 100 | 2 | 15 pmol 5′-bio Bantam |
| S5 + Dicer | | | | | 5 pmol 5′-bio Bantam |
| S6 | 0.5 | 0.1 | 100 | 2 | 15 pmol 5′-bio Bantam |
| S6 + Dicer | | 0.05 | | | 5 pmol 5′-bio Bantam |
| S7 | 0.1 | 0.005 | 100 | 2 | 2 pmol 5′-bio Bantam |
| S7 + Dicer | | | | | |
| S8 | 0.1 | 0.01 | 100 | 2 | 5 pmol 5′-bio Bantam |
| S8 + Dicer | | | | | 1 U Dicer |
| S9 | 0.1 | 0.005 | 100 | 2 | 10 pmol 5′-bio Bantam |
| S9 + Dicer | | | | | 1 U Dicer |
| S10 | 0.05 | 0.005 | 100 | 2 | 5 pmol 5′-bio Bantam |
| S10 + Dicer | | | | | 1.5 U Dicer |

(5′-bio: 5′-biotinylated)

Selection for Bantam aptamers.

| | | 5′-bio | | | |
|---|---|---|---|---|---|
| selection | RNA pool | Bantam | incubation | wash | |
| round | [uM] | [uM] | volume | [100 uL] | counter selection |
| S1 | 10 | 0.3 | 600 | 0 | streptavidin |
| S2 | 6 | 0.3 | 300 | 1 | streptavidin |
| S3 | 4 | 0.3 | 200 | 2 | streptavidin |
| S4 | 2 | 0.15 | 100 | 2 | streptavidin |
| S5 | 1 | 0.15 | 100 | 2 | 15 pmol 5′-bio pre-let-7 |
| S6 | 0.5 | 0.1 | 100 | 2 | 15 pmol 5′-bio pre-let-7 |
| S7 | 0.25 | 0.05 | 100 | 2 | 0.5 U Dicer 1 h |
| S7 + Dicer | | | | | |
| S8 | 0.1 | 0.01 | 100 | 2 | 1 U Dicer 1.5 h |
| S8 + Dicer | | | | | |
| S9 | 0.1 | 0.005 | 100 | 2 | 5 pmol 5′-bio pre-let-7 |
| S9 + Dicer | | | | | 1 U Dicer 1.5 h |
| S10 | 0.05 | 0.005 | 100 | 2 | 5 pmol 5′-bio pre-let-7 |
| S10 + Dicer | | | | | 1 U Dicer 1.5 h |

Eluted RNA molecules were ethanol precipitated. In a RT-PCR reaction total RNA molecules of one selection round were amplified with forward primer (5′-TCTAATACGACTCACTATAGGGAGAGACAAGCTTGGGTC-3′) and reverse primer (5′-ACTCCTAGGAAGAGCAAGAG-3′) in 50 uL with a Qiagen OneStep RT-PCR Kit. PCR products were phenol-chloroform extracted and ethanol precipitated. Usually 25% of PCR products were transcribed in a 100 uL reaction at 37° C. for 7 h. After removal of DNA, transcripts were phenol-chloroform extracted, ethanol precipitated and quantified by absorbance at 260 nm.

Selected DNA molecules were amplified with NEB Phusion High-Fidelity PCR Kit using primers including sequences for restriction endonucleases BamHI and EcoRI (PBam: 5′-GCTTGGATCCTCTAATACGACTCACTATAGG-3′ and PEco: 5′-GGTCGAATTCACTCCTAGGAAGAGCAAGAG-3′). Thus generated amplicons and pBSK-vector were digested with BamHI and EcoRI for 2 h at 37° C. The linearized vector and inserts were ligated in a 1:60 ratio in a 20 uL reaction. Afterwards, SURE2 cells were transformed with 5 uL of the afore-mentioned ligation reaction. Individual clones were sequenced.

Selection round S6 of pre-let-7 with Dicer already revealed RNA aptamers which inhibit Dicer cleavage up to 70% after 30 min at a concentration of 100 nM in the fluorescence assay.

Experimental Section Relating to miRNA Maturation Effectors

### Materials and General Procedures

All reagents were purchased in the highest quality available from Sigma-Aldrich or Acros Organics.

All organic reactions except the Cu-catalyzed azide-alkyne cycloaddition reactions and the synthesis of the triazide spacer were performed as described previously. The tris((1-benzyl-1H-1,2,3-triazol-4-yl)methyl)amine (TBTA) ligand used for the cyclo-addition reactions was a kind gift from Stefan Hecht (Berlin).

Fluorescence measurements were done on a BMG Labtech Fluorostar Optima plate reader. All reagents used were of highest quality available and, when possible, certified RNAse free. Water was purified with a Milli-Q® Ultrapure Water Purification System (Millipore Corp.). All buffers were additionally sterile filtered through 0.22 μM filters. Recombinant human Dicer was purchased from Invitrogen. The assay was performed as described. All the reagents including the Dicer nuclease were pipetted at 4° C. The fluorescence measurement started after a 20 minute incubation period at 37° C.

The values shown represent the mean of two measurements. Inhibition is defined by the formula 1−(I_{I}/I_{S}) after 60 minutes reaction time. I_{I} is the absolute increase of fluorescence intensity of the inhibited reaction, I_{S} is the absolute increase of fluorescence intensity of the untreated standard reaction.

SPR experiments were performed on a Biacore™ 2000 instrument in DB buffer (20 mM Tris-HCl pH 6.8, 12.5 mM NaCl, 2.5 mM MgCl₂, 1 mM DTT). 5′-Biotinylated let-7 was renatured and immobilized onto a streptavidin-coated sensor chip (SA chip, Biacore™). Conjugates were serially diluted in DB buffer (running buffer) to 300 μM, 150 μM, 75 μM, 37.5 μM, 18.75 μM, 9.38 μM, 4.69 μM, 2.34 μM and injected with the KINJECT command at 25° C. at a flow rate of 30 μl min⁻¹ for 2 min. For conjugate 2 the association time was 3 min at a flowrate of 20 μl min⁻¹. Any remaining bound conjugates after a 10 min dissociation were removed by successive injections of 10 μl 2 M NaCl, 15 μl H₂O, 10 μl 2 M NaCl and 15 μl buffer at a flow rate of 10 μl min⁻¹. Measurements were performed in duplicate. An unmodified sensor chip surface served as a reference. Data were processed with the Biacore™ evaluation software using a 1:1 Langmuir interaction model.

### Syntheses

### Synthesis of Azides

### 4-Azidophenyl sulfone

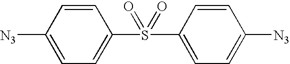

A solution of NaNO₂ (0.48 g, 7.00 mmol) in water was added dropwise to a solution of 4-aminophenyl sulfone (0.83 g, 3.33 mmol) in 2 N HCl (14 mL) at 0-5° C. with vigorous stirring. The mixture was kept below 5° C. for 30 min followed by neutralization with calcium carbonate. Then, a solution of NaN₃ (0.49 g, 7.53 mmol) in water (3 mL) was added dropwise while the temperature was kept below 5° C. The solid precipitate was filtered, washed with water (3×30 mL) and finally recrystallized from ethanol. Yield: 2.73 mmol, 82%, yellow solid.

¹H: [300 MHz, CDCl₃] δ=7.10-7.13 (m, 4H), 7.89-7.91 (m, 4H) ppm. ¹³C: [75 MHz, CDCl₃] δ=119.7, 129.5, 137.6, 145.4 ppm. MS-EI: [C₁₁H₈N₆O₂S] m/z cal. [M]^{+•}=300.0429, m/z found [M]^{+•}=300.0429.

### 1,3,5-Tris(azidomethyl)benzene

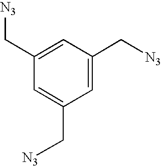

NaN₃ (0.82 g, 12.6 mmol) and 1,3,5-tris(bromomethyl)benzene (1.00 g, 2.80 mmol) were suspended in dry dimethylformamide (15 ml). After adding a catalytic amount of dicyclohexano-18-crown-6 the suspension was stirred for 72 h at 40° C. and then quenched with water (100 ml). The aqueous phase was extracted with diethyl ether (3×100 ml) and the combined extracts were re-extracted with water (3×50 mL). The combined organic layers were dried over MgSO₄ and evaporated. Yield 2.51 mmol, 90% yellowish oil.

¹H: [300 MHz, CDCl₃] δ=4.44 (s, 6H), 7.33 (s, 3H) ppm. ¹³C: [75 MHz, CDCl₃] δ=54.6, 128.8, 138.2 ppm. MS-EI: [C₉H₉N₉] m/z cal. [M]^{+•}=243.0981 m/z found [M]^{+•}=243.0981.

### 1,3-Bis(azidomethyl)benzene

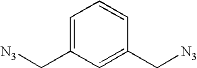

NaN₃ (1.16 g, 17.8 mmol) and 1,3-bis(bromomethyl)benzene (1.87, 7.08 mmol) were suspended in dry dimethylformamide (10 mL) and stirred at 60° C. for 12 h. Then, water (100 mL) was added and the product was extracted with diethyl ether (3×10 mL). The extracts were dried over MgSO₄ and the compound was purified by chromatography on silica gel (100% cyclohexane) to give the pure product. Yield: 5.35 mmol, 75% yellowish oil.

¹H: [300 MHz, CDCl₃] δ=4.37 (s, 4H), 7.29-7.31 (m, 3H), 7.39-7.41 (m, 1H) ppm. ¹³C: [75 MHz, CDCl₃] δ=54.5, 127.7, 127.9, 129.3, 136.1 ppm. MS-EI: [C₈H₈N₆] m/z cal. [M]^{+•}=188.0810 m/z found [M]^{+•}=188.0811.

### 1,10-Bisazidodecane

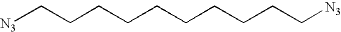

NaN₃ (1.30 g, 20.0 mmol) was suspended in a solution of 1,10-dibromodecane (2.42 g, 10.0 mmol) in dry dimethylformamide (15 mL). After stirring for 12 h at 60° C. water (15 mL) was added and the mixture was extracted with diethyl ether (3×100 mL). The organic layer was washed with water (3×50 mL), dried over Na₂SO₄ and evaporated. The crude product was purified by chromatography on silica gel (0%→20% ethyl acetate in cyclohexane). Yield: 6.82 mmol, 68% yellowish oil.

¹H: [300 MHz, CDCl₃] δ=1.30-1.54 (m, 12H), 1.51-1.68 (m, 4H), 3.26-3.35 (m, 4H) ppm. HRMS: [C₁₀H₂₀N₆] m/z cal. [M+H]⁺=225.1822 m/z found [M+H]⁺=225.1821.

### 1,11-Bisazidoundecane

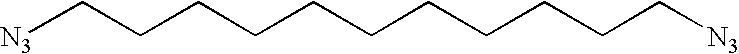

A suspension of 1,11-dibromoundecan (0.50 g, 1.59 mmol) and NaN₃ (0.31 g, 4.78 mmol) in dry dimethylformamide (15 mL) was stirred at 60° C. for 15 h. Then, water (100 mL) was added and the solution was extracted with diethyl ether (3×30 mL). The combined organic layers were washed with water (3×20 mL), dried over Na₂SO₄ and concentrated under reduced pressure to provide a residue that was of sufficient purity to use directly in the next step. Yield: 1.34 mmol, 84% yellowish oil.

¹H: [300 MHz, CDCl₃] δ=1.23-1.40 (m, 14H), 1.54-1.64 (m, 4H), 3.25 (t, 4H, J=6.9 Hz) ppm. ¹³C: [75 MHz, CDCl₃] δ=26.7, 28.8, 29.1, 29.4, 29.4, 51.4 ppm. MS-EI: [C₁₁H₂₂N₆] m/z=56.1 ([M-C₁₀H₂₀N₃]⁺, 79%), 70.1 ([M-C₉H₁₈N₃]⁺, 100%), 84.1 ([M-C₈H₁₆N₃]⁺, 30%), 98.1 ([M-C₇H₁₄N₃]⁺, 9%).

### N,N′-Bis(azidoacetyl)-1,10-diaminodecane

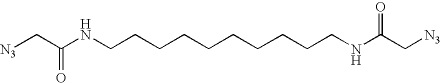

Diaminodecan (1.73 g, 10.0 mmol) was dissolved in dry toluene (15 mL) and dry triethylamine (2 mL) was added. After cooling to 0° C., Chlor acetylchloride (2.80 g, 1.97 mL, 25.0 mmol) was added dropwise over 20 min and the reaction mixture was stirred for 16 h at room temperature. The precipitate was washed with toluene (3×30 mL) and recrystallized from dimethylformamide and water. The solid (1.60 g, 4.90 mmol) was collected, dried and dissolved in dry dimethylformamide (11 mL). Then NaN₃ (1.21 g, 18.6 mmol) was added and the mixture was stirred at 60° C. over night. After completion of the reaction, indicated by TLC, water (100 mL) was added and the solid product was filtered, washed with water (3×50 mL) and recrystallized from ethanol. Yield: 3.80 mmol, 38% brown solid.

¹H: [300 MHz, CDCl₃] δ=1.24-1.38 (m, 12H), 1.49-1.54 (m, 4H), 3.27 (dd, 4H, J=6.6, 13.6 Hz), 3.98 (s, 4H), 6.32 (br, 2H) ppm. ¹³C: [75 MHz, CDCl₃] δ=26.7, 29.1, 29.3, 29.4, 39.4, 52.8, 166.4 ppm. MS-EI: [C₁₄H₂₆O₂N₈] m/z cal. [M]^{+•}=339.2251 m/z found [M]^{+•}=339.2252.

### Synthesis of Alkyne Derivatives

### Pent-4-in-1-yl p-toluene sulfonate

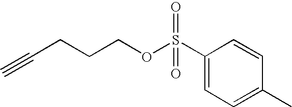

To a solution of tosyl chloride (3.84 g, 20.2 mmol), 4-pentynol (1.36 g, 1.50 mL, 16.1 mmol) and dry diethyl ether (28 mL), fresh powdered KOH (5.64 g, 101 mmol) was added over a period of 30 min with cooling between −10° C. and −20° C. Stirring was continued for 2 h at 0° C. Then the reaction was poured into icewater (50 mL) and the aqueous layer was extracted with diethyl ether (3×100 mL). The combined organic layers were dried over MgSO₄ and concentrated in vacuo to provide the crude product, which was purified by chromatography on silica gel (0%→20% ethyl acetate in cyclohexane). Yield: 11.7 mmol, 73% colourless oil.

¹H: [300 MHz, CD₃CN] δ=1.75-1.83 (m, 2H), 2.11 (t, 1H, J=2.7 Hz), 2.20 (dt, 2H, J=2.6, 7.0 Hz), 2.44 (s, 3H), 4.09 (t, 2H, J=6.1 Hz), 7.44 and 7.78 (m, 4H) ppm. ¹³C: [75 MHz, CD₃CN] δ=14.9, 21.5, 28.3, 70.2, 70.5, 83.3, 128.7, 130.9, 133.6, 46.3 ppm. HRMS: [C₁₂H₁₄O₃S] m/z cal. [M+H]⁺=261.0556 m/z found [M+H]⁺=261.0556.

### Pent-4-in-1-yl methane sulfonate

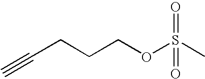

To a solution of 4-pentynol (0.50 g, 0.55 mL, 5.94 mmol) and dry methylene chloride (4.5 mL) dry triethylamine (8 mL) was added. After cooling with an acetone/dry-ice bath mesyl chloride (0.85 g, 0.57 mL, 7.42 mmol) dissolved in dry methylene chloride (2 mL) was added dropwise. The acetone/dry-ice bath was removed and the solution was stirred for additional 20 h at room temperature. The reaction mixture was poured into icewater (50 mL), washed with water (1×30 mL), HCl (2×30 mL, 1 N), dried over MgSO₄ and evaporated. Purification by chromatography on silica gel (0%→50% ethyl acetate in cyclohexane) gave the pure product. Yield: 4.60 mmol, 78% yellowish oil.

¹H: [300 MHz, CDCl₃] δ=1.92-2.00 (m, 2H), 2.11 (t, 1H, J=2.7 Hz), 2.34 (dt, 2H, J=2.7, 6.8 Hz), 3.03 (s, 3H), 4.36 (t, 2H, J=6.1 Hz) ppm. ¹³C: [75 MHz, CDCl₃] δ=14.7, 27.8, 37.2, 68.2, 69.8, 82.1 ppm. HRMS: [C₆H₁₀O₃S] m/z cal. [M+H]⁺=185.0243 m/z found [M+H]⁺=185.0242.

### Prop-2-yn-1-ol methane sulfonate

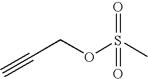

A solution of prop-2-in-1-ol (1.00 g, 1.05 mL, 17.8 mmol) in dry methylene chloride (25 mL) and dry triethylamine (4 mL) was cooled to 0° C. Then, mesyl chloride (2.66 g, 1.79 mL, 23.2 mmol) was slowly added and the reaction was allowed to stir for 3 h at 0° C. HCl (20 mL, 1 N) was then added and the aqueous layer was extracted with methylene chloride (3×100 mL). The combined organic layers were washed with sat. NaHCO₃ (50 mL), sat. NaCl (50 mL) and the aqueous layers were re-extracted with methylene chloride (3×100 mL). After drying over MgSO₄, methylene chloride was evaporated and the residue purified by chromatography on silica gel (0%→50% ethyl acetate in cyclohexane). Yield: 16.2 mmol, 91% colourless oil.

¹H: [300 MHz, CDCl₃] δ=2.70 (t, 1H, J=2.5 Hz), 3.14 (s, 3H), 4.90 (d, 2H, J=4.9 Hz) ppm. ¹³C: [75 MHz, CDCl₃] δ=38.1, 57.2, 75.8, 77.9 ppm. HRMS: [C₄H₆O₃S] m/z cal. [M+Na]⁺=156.9930 m/z found [M+Na]⁺=156.9929.

### Synthesis of 2-Deoxystreptaminderivatives

### Neamine hydrochloride

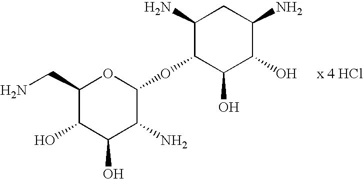

Neomycin sulfate (20.0 g, 22.0 mmol) was dissolved in methanol (500 mL) and heated to reflux. HCl (12.2 mL, 12.1 N) was then slowly added and the mixture was refluxed for additional 28 h. The solvents were removed in vacuo to give a precipitate that was filtered and washed with cold diethyl ether (3×50 mL). Yield: 17.3 mmol, 79% yellowish solid.

[α]²²_{D}: +73.1 (c 1.0, water). ¹H: [300 MHz, D₂O] δ=1.93 (q, 1H, J=12.6 Hz), 2.52 (td, 1H, J=4.2, 12.5 Hz), 3.25-3.71 (m, 6H), 4.00-4.06 (m, 3H), 5.94 (d, 1H, J=3.9 Hz) ppm. ¹³C: [75 MHz, D₂O] δ=33.3, 39.7, 47.6, 48.8, 53.5, 69.8, 69.9, 71.7, 74.4, 75.3, 84.2, 98.6 ppm. HRMS: C₁₂H₂₇N₄O₆] m/z cal. [M+H]⁺=323.1925 m/z found [M+H]⁺=323.1922.

### 1,3,2′,6′-Tetraazido neamine

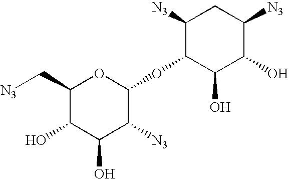

For the preparation of triflyl azide, NaN₃ (25.4 g, 388 mmol) was suspended in a mixture of methylene chloride (100 mL) and water (100 mL) and cooled to 0° C. Afterwards triflic anhydride (56.1 g, 33.0 mL, 199 mmol) was added dropwise. The icebath was removed and the reaction mixture was stirred for 2 h at room temperature. Careful quenching with sat. NaHCO₃ and extraction of the aqueous layers with methylene chloride (100 mL) provided the diazotransfer reagent, that was directly used in the next step. Then, methanol (600 mL) and a mixture of Copper-(II)-sulfate (0.25 g, 1.57 mmol) in triethylamine (20 mL), methanol (15 mL) and water (15 mL) were added to a solution of neamine hydrochloride (7.58 g, 16.3 mmol) in water (200 mL). The fresh prepared triflyl azide solution was added over a period of 20 minutes and the reaction was stirred for 20 h at room temperature. Quenching with solid NaHCO₃ (20 g) and the removal of organic solvents provided an aqueous layer that was extracted with ethyl acetate (2×200 mL). The organic layers were dried over Na₂SO₄ and concentrated to a green oil (Warning: TfN₃ has been reported to be explosive when not in solvent). Purification by chromatography on silica gel (0%→70% ethyl acetate in cyclohexane) gave the desired product. Yield: 12.5 mmol, 77% colourless solid.

[α]²²_{D}: +119.5 (c 1.0, ethyl acetate). ¹H: [300 MHz, CD₃OD] δ=1.38 (dd, 1H, J=12.2, 24.8 Hz), 2.22 (td, 1H, J=4.1, 13.1 Hz), 3.09 (dd, 1H, J=3.8, 10.5 Hz), 3.19-3.26 and 3.32-3.52 (m, 8H), 3.83 (dd, 1H, J=8.8, 10.5 Hz), 4.14 (ddd, 1H, J=2.5, 5.4, 9.9 Hz), 5.61 (d, 1H, J=3.7 Hz) ppm. ¹³C: [75 MHz, CD₃OD] δ=33.2, 52.6, 60.9, 61.8, 64.7, 72.3, 72.7, 73.3, 78.0. 78.1, 81.1, 99.7 ppm. HRMS: [C₁₂H₁₈N₁₂O₆] m/z cal. [2M+Na]⁺=875.2837 m/z found [2M+Na]⁺=875.2864.

### 1,3,2′,6′-Tetraazido-5,6-O-cyclohexylidene neamine

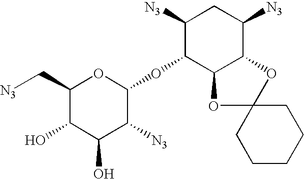

To a solution of 1,3,2′,6′-Tetraazido neamine (1.47 g, 3.45 mmol) in dry dimethylformamide, (2.5 mL) 1,1-Dimethoxycyclohexane (2.98 g, 3.15 mL, 20.7 mmol) and p-toluene sulfonic acid monohydrate (cat. amount) were added. The reaction was heated to 50° C. and 25 mbar for 5 h at a rotary evaporator and then quenched by addition of triethylamine (1 mL). After evaporation the yellow oil was dissolved in chloroform (30 mL) and washed consecutively with water (10 mL) und sat. NaHCO₃ (10 mL). The organic layer was dried over MgSO₄ and the solvent was removed in vacuo. Finally the 1,3,2′,6′-Tetraazido-3′4′;5,6-di-O-cyclohexylidene neamine (R_{f} 0.79 (50% ethyl acetate in cyclohexane)) was fully converted into the desired 1,3,2′,6′-Tetraazido-5,6-O-cyclohexyliden neamine by the following procedure: Dry dimethylformamide (25 mL), p-toluene sulfonic acid monohydrate (cat. amount) and dry methanol (0.7 mL) were added to 1,3,2′,6′-Tetraazido-3′,4′;5,6-di-O-cyclohexylidene neamine. This solution was heated for 8 h at a rotary evaporator (50° C., 25 mbar) and quenched with triethylamine (1 mL). After evaporation, the crude product was purified by chromatography on silica gel (25%→33% ethyl acetate in cyclohexane). Yield: 2.94 mmol, 85% colourless oil.

[α]²²_{D}: +93.7 (c 1.0, chloroform). ¹H: [300 MHz, CDCl₃] δ=1.36-1.55 (m, 3H), 1.57-1.70 (m, 8H), 2.32 (td, 1H, J=5.0, 13.3 Hz), 3.25 (dd, 1H, J=3.6, 10.5 Hz), 3.42 (m, 1H), 3.47-3.61 (m, 5H), 3.62-3.69 (m, 1H), 3.79-3.85 (m, 1H), 3.90-4.02 (m, 2H), 5.48 (d, 1H, J=3.6 Hz) ppm. ¹³C: [75 MHz, CD₃CN] δ=2×23.6, 24.7, 33.6, 35.9, 36.1, 51.0, 57.0, 60.7, 62.4, 70.9, 71.0, 71.2, 76.9, 79.1, 79.2, 96.1, 113.5 ppm. HRMS: [C₁₈H₂₆N₁₂O₆] m/z cal. [M+NH₄]⁺=524.2437 m/z found [M+NH₄]⁺=524.2454.

### 1,3-Diazido-5,6-O-cyclohexylidene-2-deoxystreptamine

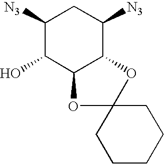

A solution of 1,3,2′,6′-Tetraazido-5,6-O-cyclohexylidene neamine (2.67 g, 5.28 mmol) in methanol (190 mL) was cooled to 0° C. After addition of NaIO₄ (8.47 g, 39.61 mmol) the reaction mixture was stirred for 15 h at room temperature. The formation of the desired bisaldehyde was monitored by TLC (R_{f} 0.74 (50% ethyl acetate in cyclohexane)). The precipitate was filtered over celite and the solution is concentrated in vacuo. The residue was dissolved in ethyl acetate (260 mL) and washed with water (260 mL) and sat. NaHCO₃ (290 mL). The organic layer was dried over Na₂SO₄ and evaporated. The solid was then dissolved in methanol (260 mL) and n-butylamine (1.15 g, 1.56 mL, 15.8 mmol) was added dropwise. After 2 h stirring at room temperature n-butylamine (0.74 g, 1 mL, 10.1 mmol) was added again and the mixture was stirred for additional 24 h. The crude product was purified by chromatography on silica gel (0%→5% ethyl acetate in cyclohexane). Yield: 4.40 mmol, 83% yellow crystals.

[α]²²_{D}: +13.3 (c 1.0, chloroform). ¹H: [300 MHz, CDCl₃] δ=1.38-1.47 (m, 3H), 1.61-1.69 (m, 8H), 2.33 (td, 1H, J=4.9, 13.6 Hz), 3.05 (br, 1H), 3.36-3.47 (m, 3H), 3.61-3.80 (m, 2H) ppm. ¹³C: [75 MHz, CDCl₃]δ=23.6, 23.7, 24.9, 33.7, 36.2, 36.2, 57.4, 62.4, 74.2, 79.1, 79.2, 113.6 ppm. HRMS: [C₁₂H₁₈N₆O₃] m/z cal. [M+H]⁺=295.1513 m/z found [M+H]⁺=295.1518.

### 1,3-Diazido-5,6-O-cyclohexylidene-4-O-pentinyl-2-deoxystreptamine

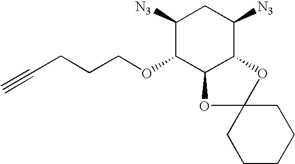

A solution of 1,3-Diazido-5,6-O-cyclohexylidene-2-deoxystreptamine (0.90 g, 3.05 mmol) in dry tetrahydrofuran (16 mL) was cooled to 0° C. Then, NaH (0.24 g, 6.10 mmol, 60% in mineral oil) was added and the suspension was stirred for 1 h. at 0° C. Then, tetrabutylammonium iodide (cat. amount) and a solution of pentinyl mesylat (0.99 g, 6.10 mmol) in tetrahydrofuran (11.5 mL) was added at 0° C. The reaction was stirred for 4 d at room temperature with repeated addition of tetrabutylammonium iodide (cat. amount) every 24 h. Then the reaction was quenched with methanol (1 mL) and poured into a mixture of icewater (30 mL) and diethyl ether (30 mL). The organic layer was washed with sat. NaHCO₃ (20 mL) and the combined aqueous layers were reextracted with diethyl ether (3×30 mL). The collected organic layers were dried over Na₂SO₄, evaporated and purified by chromatography on silica gel (0%→5% ethyl acetate in cyclohexane). Yield: 2.65 mmol, 87% yellowish oil.

[α]²²_{D}: −13.1 (c 1.0, chloroform). ¹H: [300 MHz, CD₃OD] δ=1.24-1.37 (m, 1H), 1.40-1.47 (m, 2H), 1.60-1.72 (m, 8H), 1.75-1.84 (m, 2H), 2.12-2.20 (m, 2H), 2.31 (dt, 2H, J=2.7, 7.1 Hz), 3.43-3.58 and 3.67-3.76 (m, 6H), 3.94-4.01 (m, 1H) ppm. ¹³C: [75 MHz, CD₃OD] δ=14.4, 23.5, 23.5, 24.7, 28.9, 33.5, 35.8, 36.1, 57.4, 61.2, 68.2, 69.4, 79.4, 79.5, 81.8, 83.2, 112.4 ppm. HRMS: [C₁₇H₂₄N₆O₃] m/z cal. [2M+H]⁺=721.3893 m/z found [2M+H]⁺=721.3902.

### 4-O-Pentinyl-2-deoxystreptamine

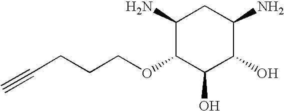

1,3-Diazido-5,6-O-cyclohexylidene-4-O-pentinyl-2-deoxystreptamine (0.60 g, 1.67 mmol) was dissolved in a mixture of dioxane (24 mL) and water (12 mL) followed by the addition of glacial acetic acid (28 mL). The mixture was stirred for 16 h at 50° C. and then concentrated in vacuo to an oil that was purified by chromatography on silica gel (10%→50% ethyl acetate in cyclohexane). The colourless oil (R_{f} 0.43 (50% ethyl acetate in cyclohexane)) was dissolved in methanol (15 mL) and NaOH (1.5 mL, 0.1 M) and trimethylphosphine (7.11 g, 8.33 mL, 8.33 mmol, 1 M in toluene) were added and the reaction was stirred for 3 h at 50° C. Finally the crude product was purified by chromatography on silica gel (0%→10% NH₄OH (28-30%) in methanol). Yield: 1.32 mmol, 79% yellowish solid.

[α]²²_{D}: +4.0 (c 1.0, methanol). ¹H: [300 MHz, CD₃OD] δ=1.25 (dd, 1H, J=12.2, 24.7 Hz), 1.74-1.90 (m, 2H), 2.00 (td, 1H, J=4.1, 12.7 Hz), 2.24 (t, 1H, J=2.6 Hz), 2.28-3.36 (m, 2H), 2.69 (ddt, 2H, J=4.1, 9.8, 12.0 Hz), 2.92 (t, 1H, J=9.4 Hz), 3.09 (t, 1H, J=9.4 Hz), 3.26 (t, 1H, J=9.1 Hz), 3.75 (ddd, 1H, J=5.8, 7.3, 9.2 Hz), 4.04 (td, 1H, J=5.9, 9.2 Hz) ppm. ¹³C: [75 MHz, CD₃OD] δ=15.9, 30.2, 36.6, 51.9, 52.5, 70.0, 72.2, 77.9, 78.9, 84.7, 87.5 ppm. HRMS: [C₁₁H₂₀N₂O₃] m/z cal. [M+H]=229.1547 m/z found [M+H]=229.1547.

### 1,3,2′,6′-Tetra-N-tert-butyloxycarbonyl neamine

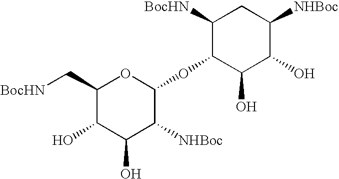

To a suspension of neamine hydrochloride (5.00 g, 10.6 mmol) in methanol (25 mL), water (5 mL) triethylamine (3 mL) and di-tert-butyl dicarbonate (22.1 g, 101 mmol) were added. The reaction was stirred at 60° C. for 1 h and then at 100° C. for further 60 min. Then the hot solution was poured into water (100 mL) and the resulting precipitate was filtrated, washed with water (3×20 mL) and dried in vacuo. Purification by chromatography on silica gel (0%→5% methanol in methylene chloride) provided the pure product. Yield: 9.26 mmol, 87% colourless solid.

[α]²²_{D}: +44.9 (c 1.0, dimethylformamide). ¹H: [300 MHz, CD₃OD] δ=1.30-1.38 (m, 1H), 1.44-1.49 (m, 36H), 2.02-2.06 (m, 1H), 3.11-3.19 (m, 1H), 3.21-3.27 and 3.35-3.58 (m, 9H), 3.71-3.74 (m, 1H), 5.26 (br, 1H) ppm. ¹³C: [75 MHz, CD₃OD] δ=6×28.8, 6×28.9, 36.1, 42.0, 51.0, 52.3, 56.9, 72.3, 72.7, 72.8, 76.5, 78.9, 80.2, 2×80.4, 80.7, 82.0, 100.4, 157.8, 158.2, 158.6, 159.3 ppm. HRMS: [C₃₂H₅₈N₄O₁₄] m/z cal. [M+H]+=723.4022 m/z found [M+H]+=723.4019.

### 1,3,2′,6′-Tetra-N-tert-butyloxycarbonyl-5,6-O-cyclohexylidene neamin

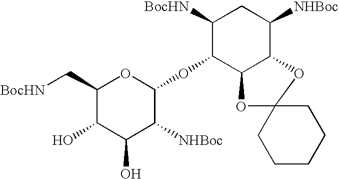

To a solution of tetra-N-tert-butyloxycarbonyl neamine (5.00 g, 6.92 mmol) in dry dimethylformamide (34 mL) 1,1-dimethoxycyclohexane (6.75 mL, 6.40 g, 44.4 mmol), and p-toluene sulfonic acid monohydrate (1.19 g, 6.26 mmol) were added. The mixture was moved on a rotary evaporator for 2 h at 50° C. and reduced pressure (25 mbar) followed by quenching with triethylamine (4 mL) and evaporation. The addition of water (10 mL) results in a white precipitate that was washed with water (3×20 mL) and dried in vacuo. 1,3,2′,6′-Tetra-N-tert-butyloxycarbonyl-3′4′;5,6-di-O-cyclohexylidene neamine (R_{f} 0.74 (4% methanol in chloroform)) was fully converted into the desired product by the following procedure: The solid was dissolved in dry dimethylformamide (40 mL) and a solution of p-toluene sulfonic acid monohydrate (0.03 g, 0.16 mmol) in dry methanol (4 mL) was added. After 60 min on the rotary evaporator (50° C., 25 mbar) the reaction was quenched with triethylamine (4 mL) und all solvents were evaporated. The residue was dissolved in chloroform (100 mL) and washed with water (30 mL) and sat. NaHCO₃ (30 mL). The organic layer was dried over MgSO₄ and evaporated to provide the crude product which was purified by chromatography on silica gel (2%→40% methanol in chloroform). Yield: 2.68 mmol, 39% colourless solid.

[α]²²_{D}: +2.6 (c 1.0, chloroform). ¹H: [300 MHz, CDCl₃] δ=1.40-1.45 (m, 39H), 1.54-1.62 (m, 8H) 2.45-2.51 (m, 1H), 3.20-3.41, 3.43-3.55 and 3.62-3.78 (m, 11H), 4.69, 4.83 and 5.06 (br, 4H), 5.30 (d, 1H, J=7.6 Hz) ppm. ¹³C: [75 MHz, CDCl₃] δ=23.7, 23.7, 24.8, 12×28.4, 36.1, 2×36.6, 41.2, 50.7, 55.0, 55.1, 71.4, 72.0, 72.0, 72.7, 72.8, 79.8, 80.0, 2×80.1, 80.2, 98.5, 112.7, 4×155.2 ppm. HRMS: [C₃₈H₆₆N₄O₁₄] m/z cal. [M+H]+=803.4648 m/z found [M+H]+=803.4644.

### 1,3-Di-N-tert-butyloxycarbonyl-5,6-O-cyclohexyliden-2-desoxystreptamin

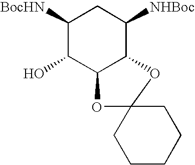

1,3,2′,6′-Tetra-N-tert-butyloxycarbonyl-5,6-O-cyclohexylidene neamine (2.00 g, 2.49 mmol) was dissolved methanol (90 mL) and cooled to 0° C. NaIO₄ (4.00 g, 18.7 mmol) was then added and the reaction was allowed to stir for 16 h at room temperature. The completion of the reaction was monitored by TLC (R_{f} 0.44 (50% ethyl acetate in cyclohexane)). The colourless precipitate was filtered over celite and the filtrate was then concentrated to dryness. The solid was dissolved in ethyl acetate (100 mL), washed with water (90 mL), sat. NaHCO₃ (90 mL), dried over MgSO₄ and concentrated in vacuo. The viscous, yellow oil was dissolved in methanol (120 mL) and n-butylamine (0.55 g, 0.74 mL, 7.48 mmol) was added dropwise. Four times the addition of n-butylamine (4×0.55 g, 0.74 mL, 7.48 mmol) was repeated every two hours, finally the solution was stirred for additional 16 h at room temperature. The solvents were evaporated and purification by chromatography on silica gel (30%→50% Ethyl acetate in cyclohexane) provided the pure product. Yield: 1.33 mmol, 53% colourless oil.

[α]²²_{D}: −23.0 (c 1.0, chloroform). ¹H: [300 MHz, CDCl₃] δ=1.37 (m, 21H), 1.52-1.64 (m, 8H), 1.92 (br, 1H), 2.40-2.58 (m, 1H), 3.31-3.37, 4.41-3.48, 3.51-3.57 and 3.63-3.72 (m, 5H), 4.74 (d, 2H, J=5.9 Hz) ppm. ¹³C: [75 MHz, CDCl₃] δ=23.6, 23.6, 24.9, 28.3, 2×36.1, 36.3, 49.2, 49.3, 73.8, 77.9, 79.5, 79.9, 80.6, 112.2, 155.3, 156.5 ppm. HRMS: [C₂₂H₃₈N₂O₇] m/z cal. [M+NH₄]⁺=460.3017 m/z found [M+NH₄]⁺=460.3021.

### 4-O-Propargyl-2-deoxystreptamine

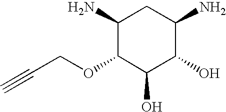

1,3-Di-N-tert-butyloxycarbonyl-5,6-O-cyclohexylidene-2-deoxystreptamine (0.50 g, 1.13 mmol) was dissolved in dry tetrahydrofuran (10 mL) and cooled to 0° C. followed by the addition of NaH (0.09 g, 2.26 mmol, 60% in mineral oil). After stirring for 1 h at 0° C. tetrabutylammonium iodide (cat. amount) and a solution of propargyl mesylat (0.30 g, 2.26 mmol) in dry tetrahydrofuran (4 mL) were added. The icebath was removed and the reaction was allowed to stir for 3 d at room temperature. The addition of tetrabutylammonium iodide (cat. amount) is repeated every 24 h. Methanol (1 mL) is used to quench the reaction which was then poured into a mixture of water (30 mL) und diethyl ether (30 mL). The aqueous layer was extracted with diethyl ether (2×30 mL) and the organic layer was washed with sat. NaHCO₃ (10 mL). All aqueous layers were re-extracted with diethyl ether (2×30 mL) and the combined organic layers were dried over Na₂SO₄ and evaporated. Purification by chromatography on silica gel (0%→30% ethyl acetate in cyclohexane) provided the intermediate product (R_{f} 0.34 (30% Ethyl acetate in cyclohexane)) which was dissolved in methylene chloride (5 mL) followed by the addition of water (0.3 mL) and trifluoracetic acid (5 mL). After 20 min the mixture was concentrated in vacuo and purified by chromatography on silica gel (0%→10% NH₄OH (28-30%) in methanol). Yield: 0.32 mmol, 28% yellowish solid.

[α]²²_{D}: +31.5 (c 1.0, water). ¹H: [300 MHz, CD₃OD] δ=1.84 (q, 1H, J=12.3 Hz), 2.43 (m, 1H), 2.99 (t, 1H, J=2.3 Hz), 3.16-3.28 (m, 2H), 3.39-3.57 (m, 3H), 4.60 (m, 2H) ppm. ¹³C: [75 MHz, CD₃OD] δ=30.0, 50.7, 51.6, 60.6, 74.4, 77.1, 77.7, 80.1, 80.7 ppm. HRMS: [C₉H₁₆N₂O₃] m/z cal. [M+H]⁺=201.1234 m/z found [M+H]⁺=201.1234.

### General procedure for the synthesis of 2-deoxystreptamine conjugates 1-9

To a solution of the alkyne-substituted 2-deoxystreptamine (33 μmol, 0.05 M in dimethylsulfoxide) and the respective diazide (15 μmol) were added 15 mol % TBTA (0.09 M solution in dimethylsulfoxide). After degassing freshly prepared sodium ascorbate solution (30 mol %, 1 M in water) was added, followed by 15 mol % of a Cu(II) sulfate solution (0.35 M in water) and the mixture was shaken for 7 days at 35° C. Then, the solution was evaporated to a minimal remainder which was purified by column chromatography on silica gel using a gradient from 0% to 15% aqueous ammonium hydroxide solution (30% w/w) in methanol. In order to separate any silica gel eluted, the obtained residue was dissolved in water (2 ml) and after centrifugation at 4° C. the supernatant was lyophilized to give the solid product. For the generation of the conjugate 9, 15 μmol of the respective triazide and 49.5 μmol of the alkyne-modified 2-deoxystreptamine were used. For all conjugates the isolated yield was 25%-30%.

¹H-NMR Data of the miRNA maturation inhibitors (conjugates) 1-9

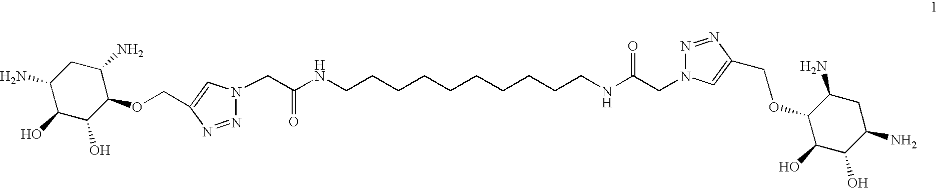

¹H-NMR [300 MHz, D₂O]:

δ=1.27-1.34 and 1.48-1.60 (m, 16H), 1.64-1.73 (m, 2H), 2.30-2.38 (m, 2H), 3.16-3.28 and 3.42-3.60 (m, 10H), 4.92 (d, 2H, J=12.1 Hz), 5.11 (d, 2H, J=12.1 Hz), 5.26 (s, 4H), 8.10 (s, 2H) ppm.

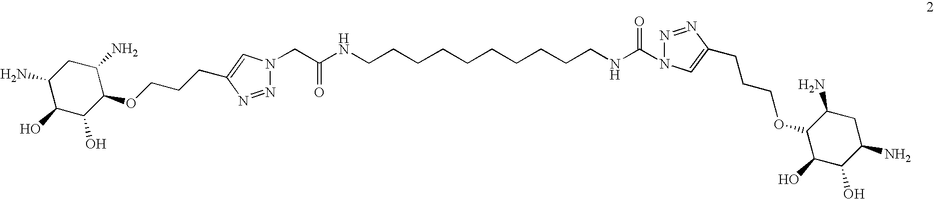

¹H-NMR [300 MHz, D₂O]:

δ=1.20-1.30 and 1.48-1.50 (m, 16H), 1.78 (q, 2H, J=12.8 Hz), 1.93-2.01 (m, 4H), 2.43 (td, 2H, J=4.3, 12.2 Hz), 2.81 (t, 4H, J=7.2 Hz), 3.20-3.34 (m, 10H), 3.49-3.51 (m, 4H), 3.70-3.75 and 3.90-3.97 (m, 4H), 5.16 (s, 4H), 7.80 (s, 2H) ppm.

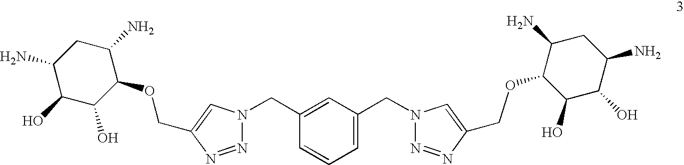

¹H-NMR [300 MHz, D₂O]:

δ=1.49-1.52 (m, 2H), 2.15-2.40 (m, 2H), 2.99-3.12 and 3.32-3.50 (m, 10H), 4.74-4.81 (m, 2H), 4.93-4.98 (m, 2H), 5.52 (s, 4H), 7.08 (br, 1H), 7.22-7.24 and 7.32-7.34 (m, 3H), 7.96 (br, 2H) ppm.

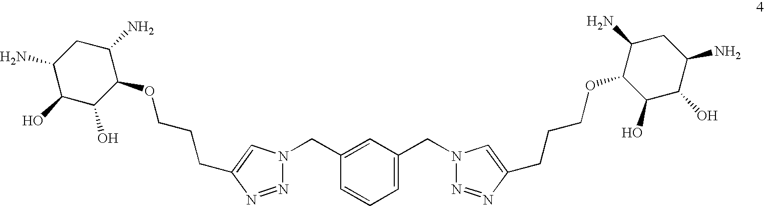

¹H-NMR [300 MHz, D₂O]:

δ=1.52-1.68 (m, 2H), 1.89-2.01 (m, 4H), 2.27-2.32 (m, 2H), 2.64 (t, 4H, J=7.4 Hz), 3.06-3.19 (m, 3H), 3.21-3.30 (m, 2H), 3.42-3.48 (m, 4H), 3.65-3.71 and 3.87-3.92 (m, 4H), 3.56 (s, 4H), 7.04 (s, 1H), 7.29-7.32 and 7.41-7.45 (m, 3H), 7.78 (s, 2H) ppm.

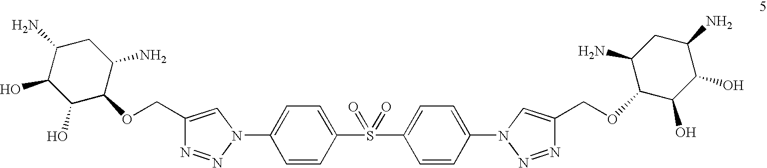

¹H-NMR [300 MHz, D₂O]:

δ=1.72-1.91 (m, 2H), 2.35 (td, 2H, J=4.1, 12.4 Hz), 3.09-3.17 and 3.41-3.62 (m, 10H), 4.98 (d, 2H, J=12.1 Hz), 5.14 (d, 2H, J=12.1 Hz), 8.05 (d, 4H, J=8.8 Hz), 8.23 (d, 4H, J=8.9 Hz), 8.62 (s, 2H) ppm.

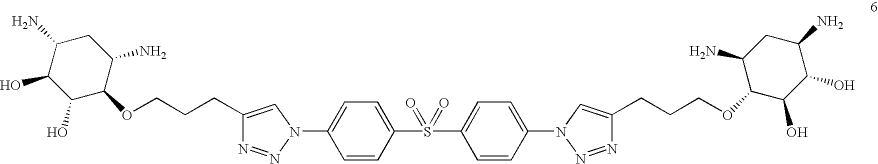

¹H-NMR [300 MHz, D₂O]:

δ=1.82 (q, 2H, J=12.4 Hz), 1.94-2.06 (m, 4H), 2.45 (td, 2H, J=4.2, 12.5 Hz), 2.85 (t, 4H, J=7.2 Hz), 3.26-3.45 (m, 6H), 3.49-3.53 (m, 4H), 3.71-3.77 and 3.94-3.99 (m, 4H), 7.99 (d, 4H, J=8.8 Hz), 8.18 (d, 4H, J=8.9 Hz), 8.33 (s, 2H) ppm.

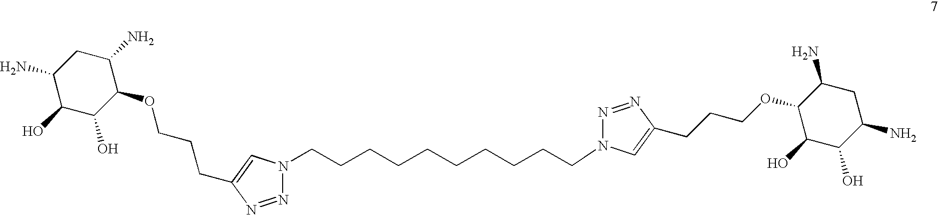

¹H-NMR [300 MHz, D₂O]:

δ=1.13-1.23 (m, 12H), 1.73-1.84 (m, 6H), 1.90-1.99 (m, 4H), 2.41-2.45 (m, 2H), 2.77 (t, 4H, J=7.1 Hz), 3.22-3.34 (m, 4H), 3.36-3.42 (m, 2H), 3.48-3.51 (m, 4H), 3.66-3.72 and 3.90-3.98 (m, 4H), 4.35 (t, 4H, J=6.8 Hz), 7.77 (s, 2H) ppm.

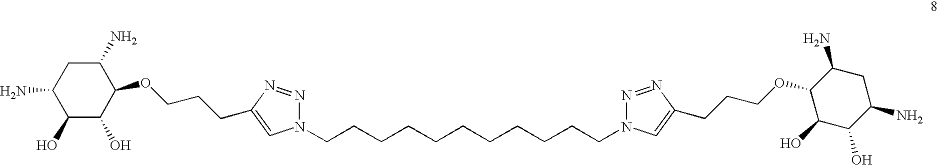

¹H-NMR [300 MHz, D₂O]:

δ=1.19 (br, 14H), 1.55-1.72 (m, 2H), 1.78-2.00 (m, 8H), 2.25-2.34 (m, 2H), 2.77 (t, 4H, J=7.3 Hz), 3.08-3.20 (m, 4H), 3.21-3.33 (m, 2H), 3.40-3.51 (m, 4H), 3.63-3.74 and 3.85-3.97 (m, 4H), 4.35 (t, 4H, J=6.8 Hz), 7.77 (s, 2H) ppm.

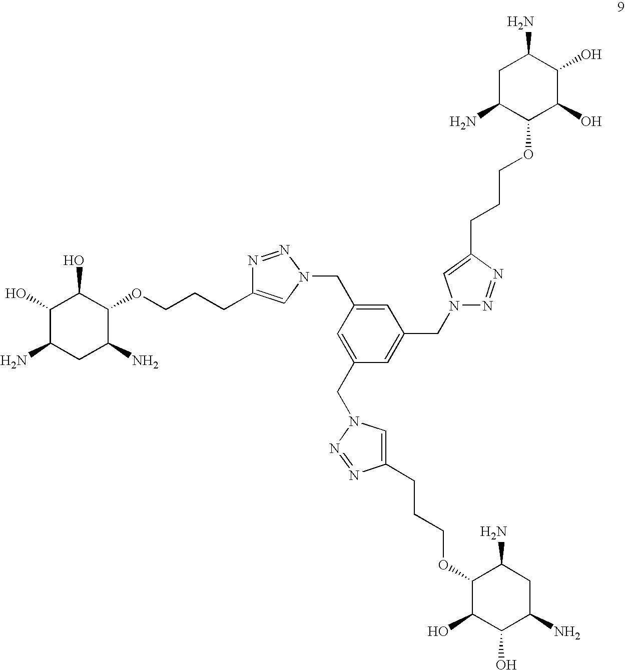

¹H-NMR [300 MHz, D₂O]:

δ=1.79-1.98 (m, 9H), 2.46 (td, 3H, J=3.6, 11.1 Hz), 2.76 (t, 4H, J=7.4 Hz), 3.21-3.38 (m, 6H), 3.39-3.47 (m, 3H), 3.49-3.57 (m, 6H), 3.65-3.74 and 3.96-3.98 (m, 6H), 5.55 (s, 6H), 7.00 (s, 3H), 7.77 (s, 3H).

We claim:

1. An assay of miRNA maturation inhibitor efficacy, comprising the steps of:
a) providing a target miRNA precursor,
b) providing a potential inhibitor of one or more processing steps of the target miRNA precursor,
c) bringing together of the target miRNA precursor and the potential inhibitor under miRNA processing conditions, and
d) determining inhibition efficacy.

2. The assay according to claim 1, wherein steps a) to d) are applied to each of a plurality of potential inhibitors.

3. The assay according to claim 1, wherein the target miRNA precursor is selected from the group consisting of pre-miRNA, pri-miRNA and Mirtrons.

4. The assay according to claim 1, wherein the processing conditions of step c) comprise provision of an enzyme with Dicer specificity.

5. The assay according to claim 1, wherein the processing conditions of step c) comprise provision of a cell expressing a miRNA processing enzyme, or a respective cell extract.

6. The assay according claim 1, further comprising the step of comparing the inhibition efficacy of a potential inhibitor with the corresponding inhibition efficacy of an aptamer according to SEQ ID NO. 8 to 15.

7. The assay according to claim 1, wherein the potential inhibitor is an RNA aptamer.

8. The assay according to claim 7, further comprising the steps of
e) selecting a potential inhibitor,
f) providing a further plurality of potential inhibitors with mutated nucleotide sequence or a modified backbone, preferably a LNA, PNA or phosphorothioate and
g) repeating steps a) to d) for each of the further plurality of potential inhibitors.

9. The assay according to any of claims 1, wherein the potential inhibitor is selected from the group consisting of peptide S1186, a peptoid, a peptide mimic, an aminoglycoside (preferably Kanamycin A), an aminoglycoside derivative, multimerized aminoglycoside derivative (preferably 2-deoxystreptamine), and combinations thereof.

10. A miRNA maturation inhibitor aptamer, comprising, consisting essentially or consisting of a nucleotide of SEQ ID NO. 8 to 15 or a nucleotide sequence having at least 70% sequence homology to any of said nucleotide sequences.

11. A miRNA maturation inhibitor, comprising, consisting essentially or consisting of an inhibitor selected from the group consisting of peptide S186, a peptoid, a peptide mimic, an aminoglycoside (preferably Kanamycin A), an aminoglycoside derivative, multimerized aminoglycoside derivative (preferably 2-deoxystreptamine), and combinations thereof.

12. A composition comprising a compound of formula (9).

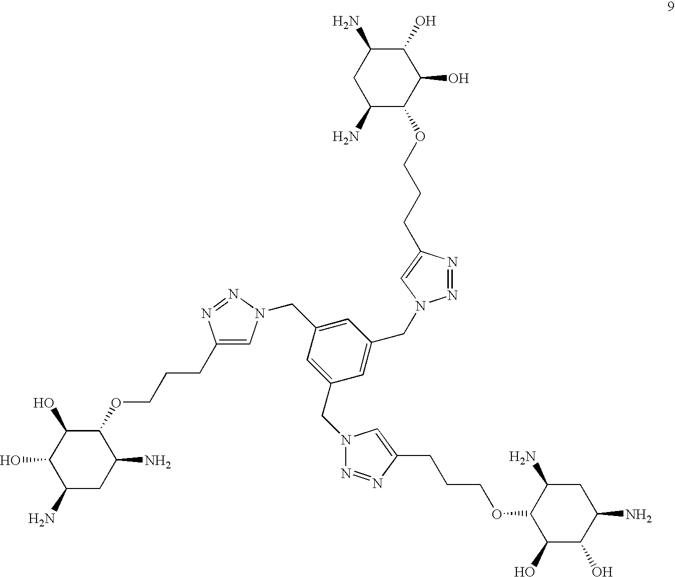

13. A miRNA maturation inhibitor composition comprising a compound of formula (X) 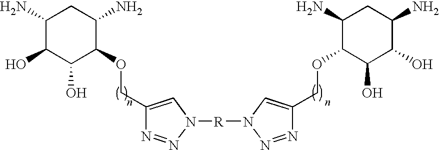
wherein R is an aliphatic or aromatic moiety having 1 to 20 carbon atoms and optionally up to 10 heteroatoms selected nitrogen, oxygen, phosphorus and sulphur, and
n is, independently of each other, 0, 1, 2, 3, 4 or 5.

14. The miRNA maturation inhibitor composition of claim 13, having a formula (1), (2), (3), (4), (5), (6), (7), (8) or (9).

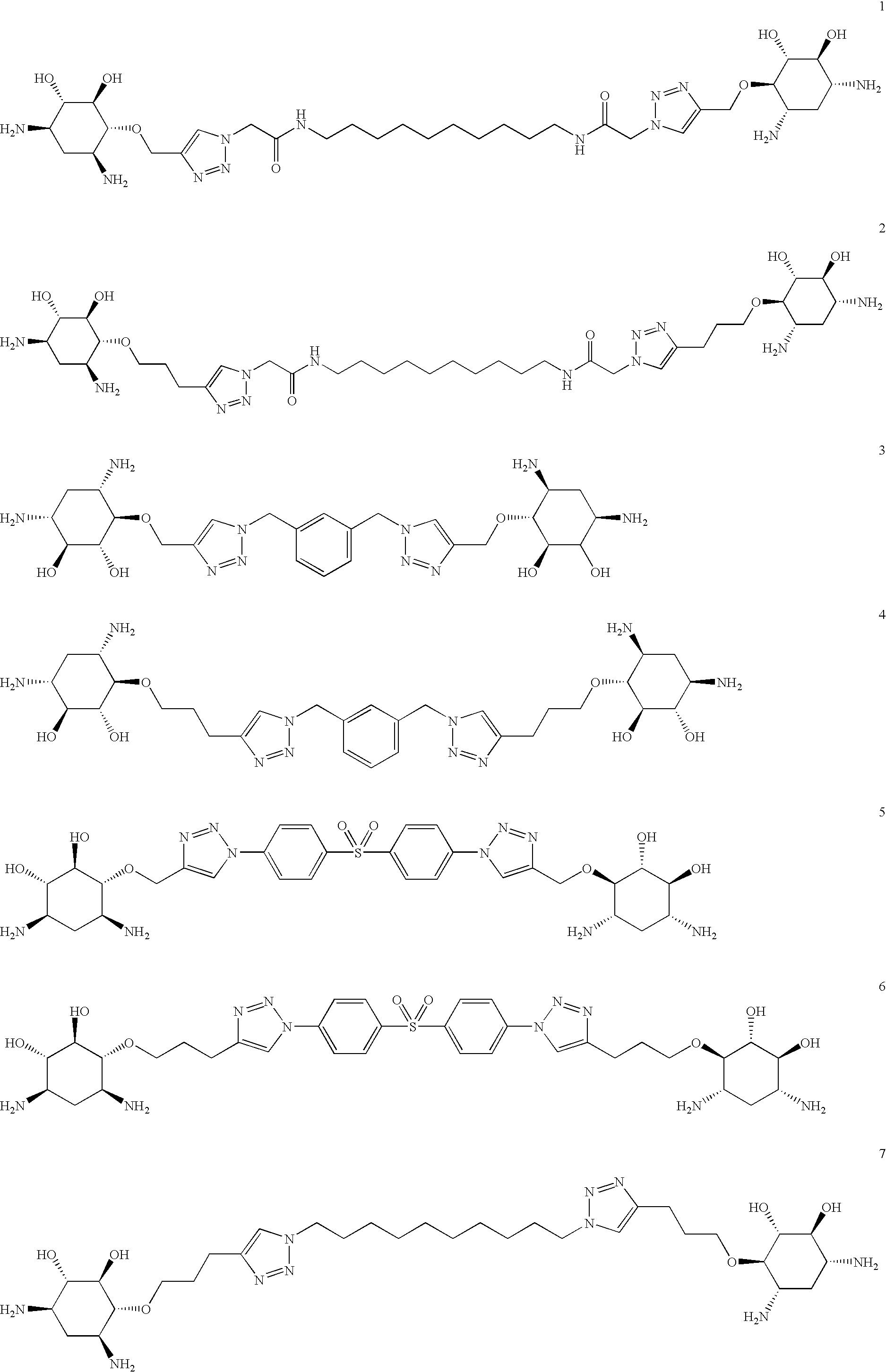

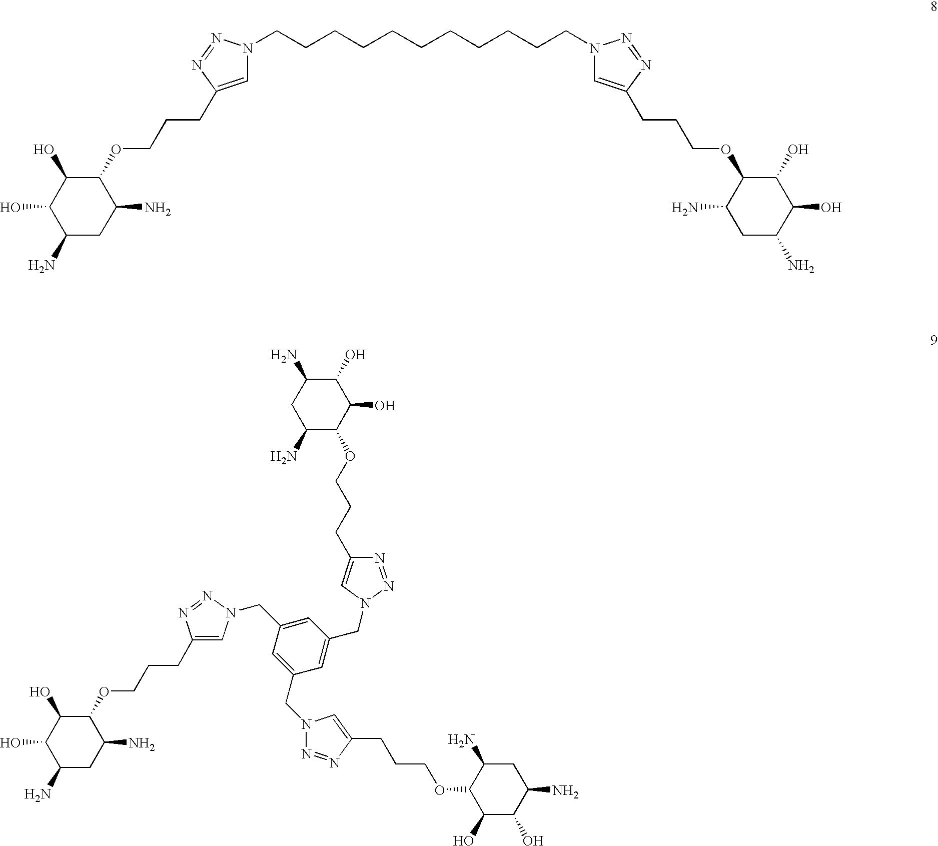

15. An assay of miRNA maturation effector efficacy, comprising the steps of
a) providing a target miRNA precursor,
b) providing a potential miRNA maturation effector, preferably a miRNA maturation inhibitor,
c) bringing together of the target miRNA precursor and the potential miRNA maturation effector under miRNA processing conditions, and determining miRNA maturation effector efficacy, and
d) bringing together of the target miRNA precursor and a miRNA maturation inhibitor of claim 13 under the miRNA processing conditions of step c), determining miRNA maturation inhibitor efficacy, and comparing the miRNA maturation effector efficacy of step c) with the miRNA maturation inhibitor efficacy of step d).
